SEQUENCE LISTING (39 sequences)

SEQ ID NO: 1 - DNA, 335 bp, Artificial Sequence
  HLP2 TRE
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 2 - DNA, 73 bp, Artificial Sequence
  Core Nucleotide Sequence
agtggacttagcccctgtttgctcctccgataactggggtgaccttggttaatattcaccagcagcctccccc

SEQ ID NO: 3 - DNA, 80 bp, Artificial Sequence
  Extended core nucleotide sequence
cccagccagtggacttagcccctgtttgctcctccgataactggggtgaccttggttaatattcaccagcagcctccccc

SEQ ID NO: 4 - DNA, 45 bp, Artificial Sequence
  5′ section of HLP2 (118-162 of SEQ ID 1)
tggacacaggacgctgtggtttctgagccagggggcgactcagat

SEQ ID NO: 5 - DNA, 41 bp, Artificial Sequence
  3′ section of HLP2 (243-283 of SEQ ID 1)
gttgcccctctggatccactgcttaaatacggacgaggaca

SEQ ID NO: 6 - DNA, 6 bp, Artificial Sequence
  TSS
tcagct

SEQ ID NO: 7 - DNA, 6 bp, Artificial Sequence
  TSS
tcaggc

SEQ ID NO: 8 - DNA, 6 bp, Artificial Sequence
  TSS
cactga

SEQ ID NO: 9 - DNA, 19 bp, Artificial Sequence
  3′ section (284-302 of SEQ ID NO 1)
gggccctgtctcctcagct

SEQ ID NO: 10 - DNA, 39 bp, Artificial Sequence
  3′ section of FRE49, FRE72 and FRE75 (297-335 of SEQ ID NO 1)
tcagcttcaggcaccaccactgacctgggacagtgaatc

SEQ ID NO: 11 - DNA, 8 bp, Artificial Sequence
  3′ section 265-272 of SEQ ID NO 1
ttaaatac

SEQ ID NO: 12 - DNA, 93 bp, Artificial Sequence
  3′ section of FRE43
gttgcccctctggatccactgcttaaatacggacgaggacagggccctgtctcctcagcttcaggcaccaccactgacctgggacagtgaatc

SEQ ID NO: 13 - DNA, 27 bp, Artificial Sequence
  3′ section of FRE56, FRE59 and FRE63
ttaaatacgggccctgtctcctcagct

SEQ ID NO: 14 - DNA, 22 bp, Artificial Sequence
  HLP2 5′ section (12-33 of SEQ ID NO 1)
gcaaacattgcaagcagcaaac

SEQ ID NO: 15 - DNA, 30 bp, Artificial Sequence
  HLP2 5′ section (12-41 of SEQ ID NO 1)
gcaaacattgcaagcagcaaacagcaaaca

SEQ ID NO: 16 - DNA, 98 bp, Artificial Sequence
  HLP2 5′ section (1-98 of SEQ ID NO 1)
ccctaaaatgggcaaacattgcaagcagcaaacagcaaacacacagccctccctgcctgctgaccttggagctggggcagaggtcagacacctctctg

SEQ ID NO: 17 - DNA, 7 bp, Artificial Sequence
  HLP2 5′ section (163-169 of SEQ ID NO 1)
cccagcc

SEQ ID NO: 18 - DNA, 98 bp, Artificial Sequence; identical to SEQ ID NO: 16
  FRE43 5′ section

SEQ ID NO: 19 - DNA, 30 bp, Artificial Sequence; identical to SEQ ID NO: 15
  FRE49 5′ section

SEQ ID NO: 20 - DNA, 81 bp, Artificial Sequence
  FRE56 5′ section
cgtgttcctgctctttgtccctctgtcctacttagactaatatttgccttgggtactgcaaacaggaaatgggggagggac

SEQ ID NO: 21 - DNA, 95 bp, Artificial Sequence
  FRE59 5′ section
gcaaacattgcaagcagcaaacagtggacttagcccctgtttgctcctccgataactggggtgaccttggttaatattcaccagcagcctccccc

SEQ ID NO: 22 - DNA, 100 bp, Artificial Sequence
  FRE63 5′ section
gcaaacattgcaagcagcaaacagtggcgtggacttagcccctgtttgctcctccgataactggggtgaccttggttaatattcaccagcagcctccccc

SEQ ID NO: 23 - DNA, 7 bp, Artificial Sequence
  FRE72 5′ section
cccagcc

SEQ ID NO: 24 - DNA, 264 bp, Artificial Sequence
  FRE43 TRE
ccctaaaatgggcaaacattgcaagcagcaaacagcaaacacacagccctccctgcctgctgaccttggagctggggcagaggtcagacacctctctgagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 25 - DNA, 149 bp, Artificial Sequence
  FRE49 TRE
gcaaacattgcaagcagcaaacagcaaacacccagccagtggacttagcccctgtttgctcctccgataactggggtgaccttggttaatattcaccagcagcctccccctcagcttcaggcaccaccactgacctgggacagtgaatc

SEQ ID NO: 26 - DNA, 181 bp, Artificial Sequence
  FRE56 TRE
cgtgttcctgctctttgtccctctgtcctacttagactaatatttgccttgggtactgcaaacaggaaatgggggagggacagtggacttagcccctgtttgctcctccgataactggggtgaccttggttaatattcaccagcagcctcccccttaaatacgggccctgtctcctcagct

SEQ ID NO: 27 - DNA, 202 bp, Artificial Sequence
  FRE59 TRE
gcaaacattgcaagcagcaaacagtggacttagcccctgtttgctcctccgataactggggtgaccttggttaatattcaccagcagcctccccccccagccagtggacttagcccctgtttgctcctccgataactggggtgaccttggttaatattcaccagcagcctcccccttaaatacgggccctgtctcctcagct

SEQ ID NO: 28 - DNA, 207 bp, Artificial Sequence
  FRE63 TRE
gcaaacattgcaagcagcaaacagtggcgtggacttagcccctgtttgctcctccgataactggggtgaccttggttaatattcaccagcagcctccccccccagccagtggacttagcccctgtttgctcctccgataactggggtgaccttggttaatattcaccagcagcctcccccttaaatacgggccctgtctcctcagct

SEQ ID NO: 29 - DNA, 119 bp, Artificial Sequence
  FRE72 TRE
cccagccagtggacttagcccctgtttgctcctccgataactggggtgaccttggttaatattcaccagcagcctccccctcagcttcaggcaccaccactgacctgggacagtgaatc

SEQ ID NO: 30 - DNA, 7 bp, Artificial Sequence
  apoE enhancer, HNF5
gcaaaca

SEQ ID NO: 31 - DNA, 4374 bp, Artificial Sequence
  co02 codop FVIII-SQ
atgcagattgagctgtctacctgcttctttctgtgcctgctgagattctgctttagtgctacaaggcgttactatctgggagctgtggagctgtcttgggattacatgcagtcagacctgggagagctgccagtggatgccagatttccccctcgagtgcccaagagcttcccttttaatacctctgtggtgtataagaaaaccctgtttgtggagtttaccgatcacctgttcaacattgctaagccaaggccaccctggatgggcctgctgggaccaacaatccaggctgaggtgtatgatacagtggtcatcaccctgaagaacatggcttcccaccctgtgtcactgcatgctgtgggagtgagctactggaaggccagtgagggagctgagtatgatgatcagaccagccagagagagaaggaggatgacaaggtgtttcctggaggctctcatacctatgtgtggcaggtgctgaaggagaatggcccaatggctagtgatcccctgtgcctgacctacagctatctgtctcatgtggacctggtgaaggatctgaacagtggcctgattggagccctgcttgtgtgtcgtgaaggctctctggccaaggaaaagacccagacactgcataagttcatcctgctttttgctgtgtttgatgagggcaagtcctggcacagtgagacaaagaactccctgatgcaggacagggatgctgccagtgccagggcctggcccaagatgcatacagtgaatggctatgtgaataggtccctgcctggcctgattggatgtcacagaaagagtgtgtattggcatgtgattggcatgggcaccacacctgaggttcactccatcttcctggagggccatacctttcttgtgagaaaccacaggcaggccagtctggagatcagtcctatcaccttcctgacagcccagaccctgcttatggatctgggccagttcctgcttttttgccacatctccagtcaccagcatgatggcatggaggcttatgtgaaggtggactcctgtcctgaggaacctcagctgagaatgaagaacaatgaggaagctgaggactatgatgatgacctgacagactctgagatggatgtggttagatttgatgatgacaactctccttcctttattcaaatccgatcagtggccaagaaacacccaaagacatgggtgcattacattgctgcagaggaggaggactgggattatgctcctctggtgctggcccctgatgacaggtcctacaagtcccagtatctgaacaatggccctcagaggattggcagaaagtacaagaaagtgaggttcatggcttatacagatgagacattcaagacaagggaggccatccagcatgagagtggcatcctgggaccactgctttatggagaagtgggagacaccctgcttatcatttttaaaaaccaggcttccaggccctacaatatctatcctcatggcatcacggatgtgagacccctgtacagtaggagactgcctaagggagtgaagcacctgaaggacttcccaatcctgcctggagagattttcaagtataagtggacagtgacagtggaggatggcccaaccaagagtgaccccaggtgcctgacaagatactattcttcctttgtgaatatggagagggacctggcctctggcctgattggacctctgcttatctgttacaaggagtctgtggatcagagaggcaaccagatcatgagtgacaagaggaatgtgatcctgttcagtgtgtttgatgagaacaggtcttggtatctgacagagaacatccagagattcctgcccaatcctgctggagtgcaactggaggaccctgagtttcaggcctccaacatcatgcatagcatcaatggctatgtgtttgactccctccaactgagtgtgtgcctgcatgaggtggcttattggtacattctgagcattggagcccagacagatttcctgagtgtgttctttagtggctacaccttcaagcataagatggtgtatgaggacaccctgacactgttccccttttctggagagacagtgttcatgtccatggagaatcctggcctgtggattctgggctgccacaactctgatttccgtaatcgtggcatgacagcccttctgaaggtgtcttcctgtgacaagaacacaggagactactatgaggattcttatgaggacatcagtgcttatctgcttagcaagaacaatgccattgagccaaggagcttttctcagaatcctccagtgctgaagagacaccagagagagatcacgcgtaccacactccagagtgatcaggaggaaattgactatgatgacacaatcagtgtggagatgaaaaaggaggactttgacatctatgatgaggatgagaaccagagccccaggtctttccagaagaaaaccagacattactttattgctgcagtggagagactgtgggattatggcatgtccagctctccacatgtgctgagaaatagagcccagagtggcagtgtgccccagttcaagaaagtggttttccaggagtttacagatggatcatttacacagcctctgtacagaggagagctgaatgagcatctgggcctgcttggcccatatatcagagctgaggtggaggataacatcatggtgaccttccgtaatcaggccagcaggccctactccttttattcatccctgatctcctatgaggaagaccagagacagggagctgagccaagaaagaactttgtgaagcccaatgagacaaagacctacttttggaaggtgcagcaccatatggcccctaccaaggatgagtttgattgcaaggcttgggcttacttcagtgatgtggatctggagaaggatgtgcattctggcctgattggaccactgcttgtgtgccataccaacacactgaatcctgctcatggcagacaagtgacagtgcaggagtttgccctgttctttaccatctttgatgagacaaagagctggtacttcacagagaacatggagaggaattgcagggctccttgtaacatccagatggaggacccaaccttcaaggagaactacagatttcatgctatcaatggctatatcatggatacactgcctggcctggtcatggctcaggaccagaggatcaggtggtatctgcttagcatgggctccaatgagaatatccacagcatccatttctctggccatgtgtttaccgtgagaaaaaaggaggaatataagatggccctgtacaacctgtatcctggagtgtttgagacagtggagatgctgccatctaaggctggcatctggagggtggagtgcctgattggagagcacctgcatgctggcatgtctaccctgtttctggtgtactccaataagtgtcagacaccactgggcatggccagtggccatatcagagatttccagatcacagcctctggacagtatggacagtgggctccaaagctggctagactgcactattctggctccatcaatgcctggtccaccaaggagcccttctcctggatcaaggtggacctgcttgctcccatgatcattcatggcatcaagacacagggagccaggcagaagttctcttccctgtacatcagccagtttatcatcatgtattctctggatggcaagaaatggcagacctacagaggcaattctacaggcacactgatggtgttctttggcaatgtggacagctctggcatcaagcacaacatcttcaatccccctatcattgctagatacatcagactgcaccctacccattattctatccgatccacactgagaatggagctgatgggctgtgatctgaacagctgttctatgccactgggcatggagtccaaggccatcagtgatgctcagatcacagcctccagctacttcaccaatatgtttgctacatggtcccctagcaaggccaggctgcacctccagggcagatccaatgcttggagacctcaagttaacaatccaaaggagtggctccaggtggattttcagaaaaccatgaaggtgacaggagtgaccacccagggagtgaagtctctgcttaccagcatgtatgtgaaggagttcctgatctcttcgagtcaagatggacaccagtggacactgttctttcagaatggcaaggtgaaggtgttccagggcaatcaggattcctttaccccagtggtgaacagcctggacccaccactgcttacaagatacctgagaatccaccctcagtcctgggtgcatcagattgctctgaggatggaggtgctgggatgtgaggctcaggacctgtattga

SEQ ID NO: 32 - DNA, 4374 bp, Artificial Sequence
  co19 codop FVIII-SQ
atgcagattgagctctccacctgcttcttcctctgcctcttgagattctgtttctctgctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagatggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtactga

SEQ ID NO: 33 - DNA, 747 bp, Artificial Sequence
  known hAAT TRE
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 34 - DNA, 4845 bp, Artificial Sequence
  FVIIIco19SQ AAV construct sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttaattaacccagccagtggacttagcccctgtttgctcctccgataactggggtgaccttggttaatattcaccagcagcctccccctcagcttcaggcaccaccactgacctgggacagtgaatcgcgccaccatgaagctgctcgcagcaactgtgctactcctcaccatctgcagccttgaaggagctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagatggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 35 - DNA, 214 bp, Artificial Sequence
  FRE75 TRE
gcaaacattgcaagcagcaaacagtggacttagcccctgtttgctcctccgataactggggtgaccttggttaatattcaccagcagcctccccccccagccagtggacttagcccctgtttgctcctccgataactggggtgaccttggttaatattcaccagcagcctccccctcagcttcaggcaccaccactgacctgggacagtgaatc

SEQ ID NO: 36 - DNA, 73 bp, Artificial Sequence; identical to SEQ ID NO: 2
  FRE46 TRE

SEQ ID NO: 37 - DNA, 79 bp, Artificial Sequence
  FRE47 TRE
agtggacttagcccctgtttgctcctccgataactggggtgaccttggttaatattcaccagcagcctccccctcagct

SEQ ID NO: 38 - DNA, 10 bp, Artificial Sequence
  transcription regulatory element ten-nucleotide termination sequence ′a′
acagtgaatc

SEQ ID NO: 39 - DNA, 10 bp, Artificial Sequence
  transcription regulatory element ten-nucleotide termination sequence ′b′
ctcctcagct

(19) United States
(12) Patent Application Publication
(10) Pub. No.: US 2022/0387558 A1
(43) Pub. Date: Dec. 8, 2022

(54) TRANSCRIPTION REGULATORY ELEMENTS

(71) Applicant: Freeline Therapeutics Limited, Stevenage (GB)

(72) Inventors: Azadeh KIA, Enfield (GB); Romuald CORBAU, Chiswell Green (GB)

(21) Appl. No.: 17/772,763

(22) PCT Filed: Oct. 30, 2020
(86) PCT No.: PCT/GB2020/052762

§ 371 Date: Apr. 28, 2022

(30) Foreign Application Priority Data
Nov. 1, 2019 (GB) 1915953.2
Nov. 1, 2019 (GB) 1915955.7
Nov. 1, 2019 (GB) 1915956.5
Dec. 6, 2019 (GB) 1917925.8
Dec. 6, 2019 (GB) 1917926.6
Dec. 6, 2019 (GB) 1917927.4
Apr. 28, 2020 (GB) 2006250.1

(51) Int. Cl. A61K 38/37; A61K 48/00; A61P 7/04; C12N 15/86

(52) U.S. Cl. CPC A61K 38/37; A61K 48/0058; A61K 48/0025; A61P 7/04; C12N 15/86; C12N 2750/14143

(57) ABSTRACT

The present invention relates to transcription regulatory elements (TREs) such as promoters, which may be used to express a transgene within a cell such as a mammalian cell. The invention further relates to polynucleotides and vectors comprising such transcription regulatory elements, which may be operably linked to a transgene, as well as methods of gene therapy based on using such vectors.

### FIELD OF THE INVENTION

The present invention relates to transcription regulatory elements (TREs) such as promoters, which may be used to express a transgene within a cell such as a mammalian cell. The invention further relates to polynucleotides and vectors comprising such transcription regulatory elements, which may be operably linked to a transgene, as well as methods of gene therapy based on using such vectors.

### BACKGROUND TO THE INVENTION

Recombinant adeno-associated virus (rAAV) vectors have considerable potential for gene therapy due to their promising safety profile and their ability to transduce many tissues in vivo. Early-stage clinical trials using such vectors have shown great promise, including long-term expression in treated patients with little to no sustained toxicity and minimal undesired immune responses.

However, as has been recognised in the art for some time (see, e.g. Chao et al. (2000) Blood, Vol. 95(5)), a particular disadvantage of rAAV vectors is their restrictive packaging capacity. In particular, the wild-type (wt) AAV has a genome of around 4.6 to 4.7 kilobases (kb), and there is evidence that rAAV genomes substantially exceeding this length lead to a heterogenous population of particles associated with sub-optimal potency and quality attributes. This can lead to problems when attempting to include transgenes for larger biological molecules: as one example, the full-length Factor VIII (FVIII) cDNA is more than 7 kb, which is too large for efficient packaging in rAAV. A truncated form of Factor VIII (known as FVIII-SQ), which lacks the central B domain and which retains its clotting efficacy, has been known for some time (Lind et al. 1995. Eur J Biochem 232, 19-27) for use e.g. in treatment of haemophilia A. However, even this truncated B-domain deleted Factor VIII cDNA is around 4.4 kb.

Previous attempts to incorporate the truncated Factor VIII (FVIII) transgene into rAAV have resulted in AAV genomes that are longer than the wild-type (Chao et al. report two constructs, both of which are longer than the wild-type 4.6 to 4.7 kb). Part of the problem lies in the fact that a functional rAAV vector requires not merely the transgene but a number of other features, such as transcription regulatory elements (promoters/enhancers), inverted terminal repeats (ITR) and polyadenylation sequences (polyA). Inclusion of all these required features has almost without exception resulted in a rAAV vector plasmid which is longer than the wild-type (wt) genome.

A number of transcription regulatory elements (promoters/enhancers) are known for use with rAAV vectors. Some known transcription regulatory elements are described in more detail in the following references: WO16/181122 (HLP2); Nathwani et al., Blood. 2006 Apr. 1, 107(7): 2653-2661 (LP1); Miao et al., Mol Ther. 2000; 1: 522-532 (HCR-hAAT); Okuyama et al., Human Gene Therapy, 7, 637-645 (1996) (ApoE-hAAT); and Wang et al., Proc Natl Acad Sci USA. 1999 March, 96(7): 3906-3910 (LSP). Such transcription regulatory elements generally comprise a promoter, an enhancer, and optionally other nucleotides.

The size (i.e. length) of the rAAV vector genome will therefore—at least in part—be defined by the size of the transcription regulatory elements, transgenes etc., and as discussed above the nature of these features will be partly or fully defined by the application for which the rAAV is to be used.

It would be desirable to be able to create rAAV vectors which are closer to the wild-type AAV genome length, including in the case of expression cassettes comprising long transgenes, and incorporating smaller transcription regulatory elements will help with this goal.

### SUMMARY OF THE INVENTION

The present invention relates to a shortened version of the known HLP2 transcription regulatory element (TRE), wherein at least some of the nucleotide sequences present in the HLP2 TRE are deleted, altered or truncated as described in more detail below. The short transcription regulatory elements of the invention are superior to the known HLP2 TRE as they have a shorter size, while retaining at least some degree of functionality, as well as potentially being associated with other surprising advantages as set out in detail below.

The present inventors have surprisingly determined that a substantial number of regions within the known HLP2 TRE can be deleted, truncated or modified without a significantly adverse impact on the efficacy of the TRE. In some instances, as exemplified below, the present inventors have surprisingly found that truncated versions (i.e. shorter versions having fewer nucleotides) of the HLP2 TRE can be made which are of comparable efficacy (i.e. around 50% or better activity by comparison) with the HLP2 TRE despite being of considerably shorter length. In some instances, the short transcription regulatory elements of the invention may have greater efficacy than HLP2 or other known transcription regulatory elements such as HCR-hAAT.

In particular, the present inventors have identified a “core” region (which may also be described as a “core sequence” or “core nucleotide sequence” or “consensus region” or “consensus nucleotide sequence”—all of these terms should be considered to be synonymous) which is present in the HLP2 TRE and which is common to all the transcription regulatory elements of the invention. As set out in detail below, the “core” region by itself can have some minimal efficacy as a TRE, but when lengthened slightly by the inclusion of further nucleotides (for example, by creating what is defined herein as an “extended core” region) the efficacy surprisingly increases considerably. The terms “extended core region” or “extended core sequence” or “extended core nucleotide sequence” or “extended consensus region” or “extended consensus nucleotide sequence” should be considered to be synonymous.

The inventors have therefore succeeded in obtaining transcription regulatory elements having reduced size (i.e., shorter) when compared to the HLP2 TRE. Having a shorter transcription regulatory element is advantageous as it allows the overall size (or length) of the rAAV genome to be reduced. This in turn allows for rAAV vectors to be made which have a genome which is closer to the wild-type genome length and thus more efficiently packaged.

It will doubtless be appreciated that there may be occasions when using a much shorter transcription regulatory element is desirable, and that a lower level of activity in respect of said transcription regulatory element can be compensated for in other ways, e.g. by using a transgene that encodes a protein having a higher level of activity with respect to the wild-type protein. One example of such is the known “Padua” Factor IX mutant having Leucine instead of Arginine at position 338 (R338L), as disclosed e.g. in WO 99/03496 (Stafford et al.).

It will therefore be understood that a transcription regulatory element having a shorter length by comparison with HLP2 is advantageous, provided that it retains at least some activity by comparison with HLP2. It will also be understood that a transcription regulatory element having both a shorter length and a greater level of activity by comparison with HLP2 is both unexpected and highly advantageous.

Accordingly, the invention provides a transcription regulatory element comprising a core nucleotide sequence which comprises or consists of a nucleotide sequence having at least 95%, or at least 98% identity to SEQ ID NO: 2, or which differs from SEQ ID NO: 2 by a single nucleotide, and wherein the transcription regulatory element is between 80 and 280 nucleotides in length; and optionally wherein the transcription regulatory element is between 80 and 225 nucleotides in length. Optionally, if a polynucleotide and/or transcription regulatory element comprises multiple copies of all or a portion of a transcription regulatory element such as a transcription regulatory element of the invention, then all of the copies should be considered to be part of the transcription regulatory element for the purpose of determining its length. For example, in a polynucleotide comprising a TRE consisting of two copies of the core region of SEQ ID NO: 2, the TRE will be considered to have a length of twice the core nucleotide sequence (i.e. 2×73 or 146 nucleotides).

In addition, the present inventors have identified specific regions of the HLP2 TRE which can be deleted, truncated or modified to create a transcription regulatory element of the invention, in the interests of minimising the size of the transcription regulatory elements and thus the overall size of the rAAV genome.

Accordingly, the present invention provides a transcription regulatory element comprising a core nucleotide sequence which comprises or consists of a nucleotide sequence having at least 95%, or at least 98% identity to SEQ ID NO: 2, or which differs from SEQ ID NO: 2 by a single nucleotide, wherein the transcription regulatory element:
a. does not comprise a nucleotide sequence according to SEQ ID NO: 4;

and/or
b. does not comprise a nucleotide sequence according to SEQ ID NO: 5;

and wherein the transcription regulatory element is between 80 and 280 nucleotides in length; and optionally wherein the transcription regulatory element is between 80 and 225 nucleotides in length.

The present invention may alternatively be defined as providing a transcription regulatory element comprising:
a. a core nucleotide sequence which comprises or consists of a nucleotide sequence having at least 95%, or at least 98% identity to SEQ ID NO: 2, or which differs from SEQ ID NO: 2 by a single nucleotide; and
b. a nucleotide sequence which is located 5′ to the core nucleotide sequence and which has less than 60% identity to a nucleotide sequence comprising at least 20, at least 25, at least 30, at least 35, at least 40 or 45 consecutive nucleotides of SEQ ID NO: 4;

wherein the transcription regulatory element is between 80 and 280 nucleotides in length; and optionally wherein the transcription regulatory element is between 80 and 225 nucleotides in length.

As will be shown below, even the transcription regulatory elements of 80 nucleotides or thereabouts show a surprising level of efficacy when compared to the HLP2 TRE. The present inventors have however discovered that the efficacy of the transcription regulatory elements of the invention can be increased by inclusion of additional nucleotide sequence(s). Such transcription regulatory elements may feature the inclusion, either 5′ or 3′ to the core nucleotide sequence, of nucleotide sequences from the HLP2 TRE or from other TREs such as the known F2 promoter/enhancer.

Accordingly the transcription regulatory element of the invention may further comprise a nucleotide sequence located 3′ to the core nucleotide sequence.

The nucleotide sequence located 3′ to the core nucleotide sequence may comprise one or more transcription start sites (TSS), which may comprise or consist of a nucleotide sequence according to:
a. SEQ ID NO: 6, or a nucleotide sequence which differs from SEQ ID NO: 6 by a single nucleotide;
b. SEQ ID NO: 7, or a nucleotide sequence which differs from SEQ ID NO:7 by a single nucleotide; and/or
c. SEQ ID NO: 8, or a nucleotide sequence which differs from SEQ ID NO: 8 by a single nucleotide.

The nucleotide sequence located 3′ to the core nucleotide sequence may comprise:
a. a nucleotide sequence according to SEQ ID NO: 6, or a nucleotide sequence which differs from SEQ ID NO: 6 by a single nucleotide; or
b. a nucleotide sequence having at least 90% identity to SEQ ID NO: 9, or a nucleotide sequence which differs from SEQ ID NO: 9 by a single nucleotide; or
c. a nucleotide sequence having at least 90% identity to SEQ ID NO: 10, or a nucleotide sequence which differs from SEQ ID NO: 10 by a single nucleotide.

The nucleotide sequence located 3′ to the core nucleotide sequence may comprise or may further comprise a nucleotide sequence defined by SEQ ID NO: 11, or a nucleotide sequence which differs from SEQ ID NO: 11 by a single nucleotide.

The nucleotide sequence located 3′ to the core nucleotide sequence may comprise a nucleotide sequence that is shorter than 50 nucleotides. It may be shorter than 40 nucleotides. It may be shorter than 30 nucleotides.

The nucleotide sequence located 3′ to the core nucleotide sequence may comprise or may consist of a nucleotide sequence selected from the group consisting of:
a. a nucleotide sequence having at least 90% identity to SEQ ID NO: 10, or a nucleotide sequence which differs from SEQ ID NO: 10 by a single nucleotide;
b. a nucleotide sequence having at least 90% identity to SEQ ID NO: 12, or a nucleotide sequence which differs from SEQ ID NO: 12 by a single nucleotide; and
c. a nucleotide sequence having at least 90% identity to SEQ ID NO: 13, or a nucleotide sequence which differs from SEQ ID NO: 13 by a single nucleotide.

A transcription regulatory element of the invention may further comprise a nucleotide sequence located 5′ to the core nucleotide sequence.

The nucleotide sequence located 5′ to the core nucleotide sequence may comprise:
a. a nucleotide sequence comprising at least 10, at least 15, or at least 20 consecutive nucleotides of SEQ ID NO: 14;
b. a nucleotide sequence having at least 90% or at least 95% identity to SEQ ID NO: 14, or a nucleotide sequence which differs from SEQ ID NO: 14 by a single nucleotide;
c. a nucleotide sequence comprising at least 10, at least 15, or at least 20 consecutive nucleotides of SEQ ID NO: 15;
d. a nucleotide sequence having at least 90% or at least 95% identity to SEQ ID NO: 15, or a nucleotide sequence which differs from SEQ ID NO: 15 by a single nucleotide;
e. a nucleotide sequence comprising at least 10, at least 15, at least 20, at least 30, at least 40, at least 50, at least 60, at least 70, at least 80 or at least 90 consecutive nucleotides of SEQ ID NO: 16;
f. a nucleotide sequence having at least 90% or at least 95% identity to SEQ ID NO: 16, or a nucleotide sequence which differs from SEQ ID NO: 16 by a single nucleotide;
   and/or
g. a nucleotide sequence defined by SEQ ID NO: 17, or a nucleotide sequence which differs from SEQ ID NO: 17 by a single nucleotide.

The nucleotide sequence located 5′ to the core nucleotide sequence may comprise an enhancer sequence, which may be defined by SEQ ID NO: 30, or a nucleotide sequence which differs from SEQ ID NO: 30 by a single nucleotide. The enhancer sequence may occur more than once as a repeat motif. It may occur twice, or three times, or more. An enhancer sequence may be directly adjacent to another enhancer sequence.

The nucleotide sequence located 5′ to the core nucleotide sequence may comprise a nucleotide sequence derived from another transcription regulatory element. The nucleotide sequence may comprise at least 10, 20, 30, 40, 50, 60, 70, or at least 80 consecutive nucleotides from another transcription regulatory element. The other transcription regulatory element may be a human transcription regulatory element. The other transcription regulatory element may be selected from the group consisting of F2 (prothrombin), alpha-1-antitrypsin, Transferrin, AM BP, Haptoglobin and transthyretin (TTR).

It is preferred that the nucleotide sequence located 5′ to the core nucleotide sequence may have less than 60% identity to a nucleotide sequence comprising at least 20, at least 25, at least 30, at least 35, at least 40 or 45 consecutive nucleotides of SEQ ID NO: 4. It may have less than 50% identity to a nucleotide sequence comprising at least 20, at least 25, at least 30, at least 35, at least 40 or 45 consecutive nucleotides of SEQ ID NO: 4. It may have less than 45% identity. It may have less than 40% identity. It may have less than 30% identity.

The nucleotide sequence located 5′ to the core nucleotide sequence may comprise a nucleotide sequence that is shorter than 110 nucleotides. It may be shorter than 100 nucleotides. It may be shorter than 50 nucleotides. It may be shorter than 10 nucleotides. It may be between 5 and 110 nucleotides in length. It may be at least 7 nucleotides in length. It may be 102 nucleotides or less in length.

The nucleotide sequence located 5′ to the core nucleotide sequence may comprise a nucleotide sequence selected from the group consisting of:
a. a nucleotide sequence having at least 90% or at least 95% identity to SEQ ID NO: 18, or a nucleotide sequence which differs from SEQ ID NO: 18 by a single nucleotide;
b. a nucleotide sequence having at least 90% or at least 95% identity to SEQ ID NO: 19, or a nucleotide sequence which differs from SEQ ID NO: 19 by a single nucleotide;
c. a nucleotide sequence having at least 90% or at least 95% identity to SEQ ID NO: 20, or a nucleotide sequence which differs from SEQ ID NO: 20 by a single nucleotide;
d. a nucleotide sequence having at least 90% or at least 95% identity to SEQ ID NO: 21, or a nucleotide sequence which differs from SEQ ID NO: 21 by a single nucleotide;
e. a nucleotide sequence having at least 90% or at least 95% identity to SEQ ID NO: 22, or a nucleotide sequence which differs from SEQ ID NO: 22 by a single nucleotide; and
f. a nucleotide sequence according to SEQ ID NO: 23, or which differs from SEQ ID NO: 23 by a single nucleotide.

It is preferred that the transcription regulatory element of the invention does not comprise:
a. a nucleotide sequence according to SEQ ID NO: 4, or does not comprise at least 20, at least 30 or at least 40 consecutive nucleotides of SEQ ID NO: 4;
   and/or
b. a nucleotide sequence according to SEQ ID NO: 5, or does not comprise at least 20, at least 30 or at least 40 consecutive nucleotides of SEQ ID NO: 5.

The transcription regulatory element of the invention may in particular not comprise:
a) a nucleotide sequence having at least 90% or at least 95% identity to SEQ ID NO: 4;
and/or
b) a nucleotide sequence having at least 90% or at least 95% identity to SEQ ID NO: 5.

The transcription regulatory element of the invention may be shorter than 200 nucleotides. It may be shorter than 150 nucleotides. It may be shorter than 125 nucleotides.

The transcription regulatory element of the invention may be at least 85 or at least 100 nucleotides in length. It may be at least 110 nucleotides in length.

The transcription regulatory element of the invention may terminate in a ten-nucleotide sequence selected from:
a. acagtgaatc; or
b. ctcctcagct.

The “core nucleotide sequence” which the present inventors have identified may be between 73 and 80 nucleotides in length. The “core nucleotide sequence” may have at least 95% identity, and optionally at least 98% identity, to SEQ ID NO: 2. The “core nucleotide sequence” may be identical to SEQ ID NO: 2.

The present inventors have identified a subset of transcription regulatory elements of the invention in which the “core nucleotide sequence” is slightly longer (referred to as the “extended core nucleotide sequence”). The “extended core nucleotide sequence” may have at least 95% identity, and optionally at least 98% identity, to SEQ ID NO: 3. The “extended core nucleotide sequence” may be identical to SEQ ID NO: 3.

The present inventors have identified particularly preferred transcription regulatory elements of the invention. These are exemplified and discussed in detail below.

The particularly preferred transcription regulatory elements of the invention may have a nucleotide sequence that has at least 90% identity, optionally at least 95% identity or optionally at least 98% identity to a nucleotide sequence selected from the group consisting of:
a. SEQ ID NO: 24
b. SEQ ID NO: 25
c. SEQ ID NO: 26
d. SEQ ID NO: 27
e. SEQ ID NO: 28; and
f. SEQ ID NO: 29

SEQ ID NO: 24 defines a transcription regulatory element of the invention (allocated the internal designation “FRE43”) having properties which are detailed in the examples below. It comprises the “core nucleotide sequence” of nucleotides 170-242 of HLP2 (as defined herein with reference to SEQ ID NO: 1), while the 5′ section lacks nucleotides 99-169 of HLP2 (and therefore completely lacks nucleotides 118-162 of HLP2) and includes nucleotides 1-98 of HLP2. The 3′ section includes nucleotides 243-335 of HLP2. The overall length of FRE43 is 264 nucleotides.

FRE43 therefore provides a transcription regulatory element of the invention which (inter alia):
comprises the core nucleotide sequence as defined by SEQ ID NO: 2;
does not comprise a nucleotide sequence according to SEQ ID NO: 4;
comprises a nucleotide sequence which is located 5′ to the core nucleotide sequence and which has less than 60% identity to SEQ ID NO: 4;
comprises a nucleotide sequence located 3′ to the core nucleotide sequence which can be defined by SEQ ID NO: 9 or SEQ ID NO: 10 and which includes 3 TSS defined by SEQ ID Nos: 6, 7 and 8; and
comprises a nucleotide sequence located 3′ to the core nucleotide sequence which further includes SEQ ID NO: 11.

It will be understood that since the 3′ section of FRE43 includes nucleotides 243-335 of HLP2, it can be defined as comprising SEQ ID NO: 9 or can be defined as comprising SEQ ID NO: 10.

FRE43 may also be defined as providing a core nucleotide sequence defined by SEQ ID NO: 2; a 5′ region defined by SEQ ID NO: 18; and a 3′ region defined by SEQ ID NO: 12.

SEQ ID NO: 25 defines a transcription regulatory element of the invention (allocated the internal designation “FRE49”) having properties which are detailed in the examples below. It comprises the “extended core nucleotide sequence” of nucleotides 163-242 of HLP2, while the 5′ section lacks nucleotides 1-11 and nucleotides 42-162 of HLP2 (and therefore completely lacks nucleotides 118-162 of HLP2) and includes nucleotides 12-41 of HLP2. The 3′ section lacks nucleotides 243-296 of HLP2 (and therefore completely lacks nucleotides 243-283 of HLP2) and includes nucleotides 297-335 of HLP2. The overall length of FRE49 is 149 nucleotides.

FRE49 therefore provides a transcription regulatory element of the invention which (inter alia):
comprises the extended core nucleotide sequence as defined by SEQ ID NO: 3;
does not comprise a nucleotide sequence according to SEQ ID NO: 4;
does not comprise a nucleotide sequence according to SEQ ID NO: 5;
comprises a nucleotide sequence which is located 5′ to the core nucleotide sequence and which has less than 60% identity to SEQ ID NO: 4; and
comprises a nucleotide sequence located 3′ to the core nucleotide sequence defined by SEQ ID NO: 10 and which includes 3 TSS defined by SEQ ID Nos: 6, 7 and 8.

FRE49 may also be defined as providing an extended core nucleotide sequence defined by SEQ ID NO: 3; a 5′ region defined by SEQ ID NO: 19; and a 3′ region defined by SEQ ID NO: 10.

SEQ ID NO: 26 defines a transcription regulatory element of the invention (allocated the internal designation “FRE56”) having properties which are detailed in the examples below. It comprises the “core nucleotide sequence” of nucleotides 170-242 of HLP2, while the 5′ section comprises a nucleotide sequence derived from the F2 TRE. The 3′ section lacks nucleotides 243-264, nucleotides 273-283 and nucleotides 303-335 of HLP2 (and therefore does not include a nucleotide sequence defined by nucleotides 243-283 of HLP2) and includes nucleotides 265-272 and 284-302 of HLP2. The overall length of FRE56 is 181 nucleotides.

FRE56 therefore provides a transcription regulatory element of the invention which (inter alia):
comprises the core nucleotide sequence as defined by SEQ ID NO: 2;
does not comprise a nucleotide sequence according to SEQ ID NO: 4;
does not comprise a nucleotide sequence according to SEQ ID NO: 5;
comprises a nucleotide sequence which is located 5′ to the core nucleotide sequence and which has less than 60% identity to SEQ ID NO: 4;
comprises a nucleotide sequence located 3′ to the core nucleotide sequence defined by SEQ ID NO: 9 and which includes a TSS defined by SEQ ID No: 6; and
comprises a nucleotide sequence located 3′ to the core nucleotide sequence which further includes SEQ ID NO: 11.

FRE56 may also be defined as providing a core nucleotide sequence defined by SEQ ID NO: 2; a 5′ region defined by SEQ ID NO: 20; and a 3′ region defined by SEQ ID NO: 13.

SEQ ID NO: 27 defines a transcription regulatory element of the invention (allocated the internal designation “FRE59”) having properties which are detailed in the examples below. It comprises the “extended core nucleotide sequence” of nucleotides 163-242 of HLP2, while the 5′ section lacks nucleotides 1-11, nucleotides 71-92, nucleotides 101-105 and nucleotides 113-133 of HLP2 (and therefore does not include a nucleotide sequence defined by nucleotides 118-162 of HLP2) and includes nucleotides 12-33 of HLP2. The 5′ section of FRE59 also includes a nucleotide sequence at least partially corresponding to nucleotides 170-242 of HLP2 (i.e., a nucleotide sequence corresponding to the ‘core region’): nucleotides 23-95 of the FRE59 sequence (SEQ ID NO: 27) are identical to nucleotides 1-73 of the core region (SEQ ID NO: 2). The 3′ section lacks nucleotides 243-264, nucleotides 273-283 and nucleotides 303-335 of HLP2 (and therefore does not include a nucleotide sequence defined by nucleotides 243-283 of HLP2) and includes nucleotides 265-272 and 284-302 of HLP2. The overall length of FRE59 is 202 nucleotides.

FRE59 therefore provides a transcription regulatory element of the invention which (inter alia):
comprises the extended core nucleotide sequence as defined by SEQ ID NO: 3;
does not comprise a nucleotide sequence according to SEQ ID NO: 4;
does not comprise a nucleotide sequence according to SEQ ID NO: 5;
comprises a nucleotide sequence which is located 5′ to the core nucleotide sequence and which has less than 60% identity to SEQ ID NO: 4;
comprises a nucleotide sequence located 3′ to the core nucleotide sequence defined by SEQ ID NO: 9 and which includes a TSS defined by SEQ ID No: 6; and
comprises a nucleotide sequence located 3′ to the core nucleotide sequence which further includes SEQ ID NO: 11.

FRE59 may also be defined as providing an extended core nucleotide sequence defined by SEQ ID NO: 3; a 5′ region defined by SEQ ID NO: 21; and a 3′ region defined by SEQ ID NO: 13. The 5′ region may be further defined as comprising a nucleotide sequence according to the core region (i.e. SEQ ID NO: 2).

SEQ ID NO: 28 defines a transcription regulatory element of the invention (allocated the internal designation “FRE63”) having properties which are detailed in the examples below. It comprises the “extended core nucleotide sequence” of nucleotides 163-242 of HLP2, while the 5′ section lacks nucleotides 1-11, nucleotides 36-72, nucleotides 99-104 and nucleotides 121-125 of HLP2 (and therefore does not include a nucleotide sequence defined by nucleotides 118-162 of HLP2) and includes nucleotides 12-33 of HLP2. The 5′ section of FRE63 also includes a nucleotide sequence at least partially corresponding to nucleotides 170-242 of HLP2 (i.e., a nucleotide sequence corresponding to the ‘core region’): a sequence comparison (not shown) demonstrates that nucleotides 29-100 of the FRE63 sequence (SEQ ID NO: 28) are identical to nucleotides 2-73 of the core region (SEQ ID NO: 2). The 3′ section lacks nucleotides 243-264, nucleotides 273-283 and nucleotides 303-335 of HLP2 (and therefore does not include a nucleotide sequence defined by nucleotides 243-283 of HLP2) and includes nucleotides 265-272 and 284-302 of HLP2. The overall length of FRE63 is 207 nucleotides.

FRE63 therefore provides a transcription regulatory element of the invention which (inter alia):
comprises the extended core nucleotide sequence as defined by SEQ ID NO: 3;
does not comprise a nucleotide sequence according to SEQ ID NO: 4;
does not comprise a nucleotide sequence according to SEQ ID NO: 5;
comprises a nucleotide sequence which is located 5′ to the core nucleotide sequence and which has less than 60% identity to SEQ ID NO: 4;
comprises a nucleotide sequence located 3′ to the core nucleotide sequence defined by SEQ ID NO: 9 and which includes a TSS defined by SEQ ID No: 6; and
comprises a nucleotide sequence located 3′ to the core nucleotide sequence which further includes SEQ ID NO: 11.

FRE63 may also be defined as providing an extended core nucleotide sequence defined by SEQ ID NO: 3; a 5′ region defined by SEQ ID NO: 22; and a 3′ region defined by SEQ ID NO: 13. The 5′ region may be further defined as comprising a partial sequence (nucleotides 2-73) of the core region (i.e. SEQ ID NO: 2).

SEQ ID NO: 29 defines a transcription regulatory element of the invention (allocated the internal designation “FRE72”) having properties which are detailed in the examples below. It comprises the “extended core nucleotide sequence” of nucleotides 163-242 of HLP2, while the 5′ section lacks nucleotides 1-162 of HLP2 (and therefore completely lacks nucleotides 118-162 of HLP2). FRE72 can therefore be defined as not having a 5′ section (i.e. as not having a nucleotide sequence that is 5′ to the “extended core nucleotide sequence”). Alternatively, if FRE72 is instead considered to have the core nucleotide sequence of nucleotides 170-242 of HLP2, then it can be considered to have a 5′ section consisting of nucleotides 163-169 of HLP2. The 3′ section lacks nucleotides 243-296 of HLP2 (and therefore completely lacks nucleotides 243-283 of HLP2) and includes nucleotides 297-335 of HLP2. The overall length of FRE72 is 119 nucleotides.

FRE72 therefore provides a transcription regulatory element of the invention which (inter alia):
comprises the extended core nucleotide sequence as defined by SEQ ID NO: 3;
does not comprise a nucleotide sequence according to SEQ ID NO: 4;
does not comprise a nucleotide sequence according to SEQ ID NO: 5;
comprises a nucleotide sequence which is located 5′ to the core nucleotide sequence and which has less than 60% identity to SEQ ID NO: 4; and
comprises a nucleotide sequence located 3′ to the core nucleotide sequence defined by SEQ ID NO: 10 and which includes 3 TSS defined by SEQ ID Nos: 6, 7 and 8.

FRE72 may also be defined as providing a core nucleotide sequence defined by SEQ ID NO: 2; a 5′ region defined by SEQ ID NO: 23; and a 3′ region defined by SEQ ID NO: 10.

Alternatively, FRE72 may be defined as providing an extended core nucleotide sequence defined by SEQ ID NO: 3; and a 3′ region defined by SEQ ID NO: 10.

In light of the above, it will be understood that where a transcription regulatory element of the invention comprises the ‘extended core’ sequence defined by SEQ ID NO: 3, the 5′ region (where there is one) may comprise a nucleotide sequence that has at least 60, at least 70 or at least 71 or 72 nucleotides of the ‘core nucleotide sequence’ defined by SEQ ID NO: 2.

Accordingly the present invention provides a transcription regulatory element of the invention comprising:
(a) an ‘extended core’ sequence which comprises or consists of a nucleotide sequence having at least 95%, or at least 98% identity to SEQ ID NO: 3; and
(b) a nucleotide sequence located 5′ to the extended core nucleotide sequence, which comprises a nucleotide sequence having at least 95% or at least 98% identity to SEQ ID NO: 2;

wherein the transcription regulatory element is between 80 and 280 nucleotides in length.

The 5′ region may comprise other elements as set out above (such as e.g. a nucleotide sequence comprising at least 10, at least 15, or at least 20 consecutive nucleotides of SEQ ID NO: 14).

The transcription regulatory element may additionally comprise a 3′ region as defined above.

It is fully envisaged that, based on the overall teaching of the present application, the skilled person will understand that individual elements from any one or more of the various above-defined transcription regulatory elements can be combined, in order to obtain one or more further transcription regulatory elements of the invention.

Accordingly, the present invention provides a transcription regulatory element according to or derived from the nucleotide sequence defined by SEQ ID NO: 1 (the HLP2 TRE), wherein the transcription regulatory element comprises:
(a) a core nucleotide sequence which comprises or consists of a nucleotide sequence having at least 95%, or at least 98% identity to:
   (i) nucleotides 170-242 numbered according to SEQ ID NO: 1;
   or
   (ii) nucleotides 163-242 numbered according to SEQ ID NO: 1;
(b) one or more deletions to the nucleotide sequence located 5′ to the core nucleotide sequence, wherein the deletions are selected from:
   (i) nucleotides 1-11, numbered according to SEQ ID NO: 1; and/or
   (ii) nucleotides 36-72, numbered according to SEQ ID NO: 1; and/or
   (iii) nucleotides 71-92, numbered according to SEQ ID NO: 1; and/or
   (iv) nucleotides 99-104, numbered according to SEQ ID NO: 1; and/or
   (v) nucleotides 101-105, numbered according to SEQ ID NO: 1; and/or
   (vi) nucleotides 121-125, numbered according to SEQ ID NO: 1; and/or
   (vii) nucleotides 42-162, numbered according to SEQ ID NO: 1; and/or
   (viii) nucleotides 113-133, numbered according to SEQ ID NO: 1; and/or
   (ix) nucleotides 1-162, numbered according to SEQ ID NO: 1;
(c) optionally one or more deletions to the nucleotide sequence located 3′ to the core nucleotide sequence, wherein the deletions are selected from:
   (i) nucleotides 243-296, numbered according to SEQ ID NO: 1; and/or
   (ii) nucleotides 243-264, numbered according to SEQ ID NO: 1; and/or
   (iii) nucleotides 273-283, numbered according to SEQ ID NO: 1; and/or
   (iv) nucleotides 303-335, numbered according to SEQ ID NO: 1;

and wherein the transcription regulatory element is between 80 and 280 nucleotides in length.

In another embodiment the invention provides a transcription regulatory element according to or derived from the nucleotide sequence defined by SEQ ID NO: 1 (the HLP2 TRE), wherein the transcription regulatory element comprises a core nucleotide sequence which comprises or consists of a nucleotide sequence having at least 95%, or at least 98% identity to nucleotides 170-242, or to nucleotides 163-242, numbered according to SEQ ID NO: 1; and has one more deletions from the nucleotide sequence defined by nucleotides 1-162 of SEQ ID NO: 1.

The one or more deletions may be from 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160 or 162 consecutive nucleotides of the nucleotide sequence defined by nucleotides 1-162 of SEQ ID NO: 1.

The transcription regulatory element may further comprise one or more deletions from the nucleotide sequence defined by nucleotides 243-335, numbered according to SEQ ID NO: 1.

The one or more deletions may be from 5, 10, 15, 20, 25, 30, 35, 40, 45, 50, 55, 60, 65, 70, 75, 80, 85 or 90 consecutive nucleotides of the nucleotide sequence defined by nucleotides 243-335 of SEQ ID NO: 1.

The one or more deletions may be from 5, 10, 15, 20, 25, 30, 35, 40, 45, 50, 55, 60, 65, 70, 75, 80, 85 or 90 consecutive nucleotides of the nucleotide sequence defined by nucleotides 243-283 of SEQ ID NO: 1.

The one or more deletions may be from 5, 10, 15, 20, 25, 30, 35, 40, 45, 50, 55, 60, 65, 70, 75, 80, 85 or 90 consecutive nucleotides of the nucleotide sequence defined by nucleotides 243-296 of SEQ ID NO: 1.

The one or more deletions may be from 5, 10, 15, 20, 25, 30, 35, 40, 45, 50, 55, 60, 65, 70, 75, 80, 85 or 90 consecutive nucleotides of the nucleotide sequence defined by nucleotides 303-335 of SEQ ID NO: 1.

The transcription regulatory element according to or derived from the nucleotide sequence defined by SEQ ID NO: 1 (the HLP2 TRE) may include a 5′ and/or a 3′ region as defined above.

The transcription regulatory element of the invention may comprise, or may be, a promoter. The promoter may be a liver-specific promoter and/or may further comprise an enhancer. Optionally, the transcription regulatory element is liver-specific. In some embodiments, the transcription regulatory element or the promoter is liver-specific if it promotes protein expression at higher levels in liver cells compared to cells from at least one other organ or tissue.

Optionally, the transcription regulatory element or the promoter is liver-specific if it promotes protein expression at higher levels in liver cells compared to cells from at least one other organ or tissue and the transcription regulatory element or the promoter promotes protein expression in the cells from at least one other organ or tissue at a level less than 40%, less than 30%, less than 25%, less than 15%, less than 10%, or less than 5% of the level that the transcription regulatory element or the promoter promotes protein expression in liver cells.

Optionally, the cells from at least one other organ or tissue are at least one of kidney cells, pancreatic cells, breast cells, neuroblastoma cells, lung cells, and early B cells. Optionally, the cells from at least one other organ or tissue are kidney cells, pancreatic cells, breast cells, neuroblastoma cells, lung cells, and early B cells. Optionally, the cells from at least one other organ or tissue are at least one of HEK293T cells, PANC1 cells, BxPC-3 cells, MCF7 cells, 1643 cells, MRC-9 cells, and 697 cells. Optionally, the cells from at least one other organ or tissue are HEK293T cells, PANC1 cells, BxPC-3 cells, MCF7 cells, 1643 cells, MRC-9 cells, and 697 cells.

Optionally, whether or not the transcription regulatory element or the promoter is liver-specific may be determined by transducing Huh7 cells and comparator cells with a vector comprising the transcription regulatory element or promoter operably linked to a transgene and comparing the number of Huh7 cells that express the transgene with the number of comparator cells that express the transgene. The comparator cells can be non-liver cells. For example, if the user wishes to determine whether the transcription regulatory element or promoter promotes expression at higher levels in liver cells compared to breast cells, the user may transduce Huh7 cells and comparator cells that are breast cells such as MCF7 cells. If the number of Huh7 cells that express the transgene is significantly higher than the number of comparator cells that express the transgene then the promoter or transcription regulatory element is liver-specific.

The transgene may be GFP, in which case the user may determine the number of cells (e.g. the number of Huh7 cells or the number of comparator cells) that express the transgene using fluorescence microscopy and counting the number of cells that fluoresce green.

The present invention further provides a polynucleotide sequence comprising a transcription regulatory element or promoter of the invention, wherein the transcription regulatory element or promoter is operably linked to a transgene, optionally wherein the transgene encodes a human protein.

The transcription regulatory element or promoter of the invention may be part of a vector comprising a transgene. The vector may be a viral particle such as an AAV vector or recombinant AAV (rAAV) vector.

The transgene may encode a protein or a non-translated RNA which may be, for example, an siRNA or miRNA or a snRNA or an antisense RNA. The transgene may be longer than 4,000 (4 k) nucleotides, or 4,000 base pairs (4 kbp). The transgene may be longer than 4.2 k nucleotides. The transgene may be shorter than 4.4 k nucleotides.

In one embodiment the transgene encodes a clotting factor, such as Factor VIII (which may be a truncated FVIII as discussed elsewhere herein) or Factor IX. The transgene may alternatively encode an enzyme, which may be a lysosomal enzyme such as alpha-galactosidase A or beta-glucocerebrosidase (GBA).

The transcription regulatory element or promoter of the invention, as well as polynucleotides comprising such elements or promoters and vectors including them, may express a transgene to which it is operably linked at 50% or better compared to the HLP2 TRE (defined by SEQ ID NO: 1) or the HCR-hAAT TRE (defined by SEQ ID NO: 33).

It may express a transgene to which it is operably linked at 80% or better compared to the HLP2 TRE (defined by SEQ ID NO: 1) or the HCR-hAAT TRE (defined by SEQ ID NO: 33).

The present inventors have surprisingly discovered not only that truncated (often significantly truncated) versions of the HLP2 TRE can be made which retain their function, but that the truncated transcription regulatory elements of the invention may in fact have a superior effect by comparison with the HLP2 TRE or the HCR-hAAT TRE.

Accordingly, the transcription regulatory element or promoter of the invention, as well as polynucleotides comprising such elements or promoters and vectors including them, may express a transgene to which it is operably linked at 100% or better, 110% or better, 120% or better, 140% or better, or 150% or better, compared to the HLP2 TRE (defined by SEQ ID NO: 1) or the HCR-hAAT TRE (defined by SEQ ID NO: 33).

The skilled person may compare the expression of a transgene using a transcription regulatory element of the invention to the HLP2 TRE (SEQ ID NO: 1) or the HCR-hAAT TRE (SEQ ID NO: 33) by comparing the level of polypeptide encoded by the transgene expressed under the control of a transcription regulatory element of the invention with the level of polypeptide encoded by the transgene expressed under the control of the HLP2 TRE or the HCR-hAAT TRE in an in vitro or an in vivo system.

For example, in an in vitro test (such as that set out in Example 2) to compare the level of polypeptide encoded by the transgene expressed, the skilled person could transduce host cells with a vector comprising the TRE of the invention operably linked to the transgene (test cells), and some cells with a vector comprising HLP2 or HCR-hAAT operably linked to the transgene (reference cells). The cells may be cultured under conditions suitable for expressing the transgene, and the level of polypeptide encoded by the transgene expressed in the test cells and reference cells can be compared. Suitable host cells include cultured human liver cells, such as Huh7 cells. The level of polypeptide should be normalised to reflect the number of cells that have been transfected using a luciferase assay. In the luciferase assays the test cells and the reference cells are also transfected using an equivalent vector (identical except for the promoter and the transgene) comprising a luciferase transgene, and the proportion of cells that are transfected by the vector will be proportionate to the fluorescent signal produced by the luciferase expressed from the vector comprising the luciferase transgene.

Similarly, to compare the level of polypeptide encoded by the transgene expressed in an in vivo system, the skilled person could inject some mice (such as C57BL/6 mice) with a viral particle comprising the TRE of the invention operably linked to the transgene (test mice), and some equivalent mice with a viral particle comprising HLP2 or HCR-hAAT operably linked to the transgene (reference mice). The mice may be culled and the level of polypeptide encoded by the transgene in the blood of the test mice may be compared to the level of polypeptide encoded by the transgene in the blood of the reference mice. The level of polypeptide encoded by the transgene may be normalised to the number of vector genomes per liver cell.

The level of polypeptide encoded by the transgene can be assessed using an ELISA. In an example of an ELISA assay, an antibody that binds to the polypeptide encoded by the transgene could be bound to a plate. The sample, comprising the polypeptide encoded by the transgene at unknown concentration, could be passed over the plate. A second detection antibody that binds to the polypeptide encoded by the transgene could be applied to the plate, and any excess washed off. The detection antibody that remains (i.e. is not washed off) will be bound to the polypeptide encoded by the transgene. The detection antibody could be linked to an enzyme such as horse radish peroxidase. The level of detection antibody that binds to the polypeptide encoded by the transgene on the plate could be measured by measuring the amount of the detection antibody. For example, if the detection antibody is linked to horse radish peroxidase, the horse radish peroxidase can catalyse the production of a blue reaction product from a substrate such as TMB (3,3′,5,5′-tetramethylbenzidine), and the level of the blue product can be detected by absorbance at 450 nm. The level of the blue product is proportional to the amount of detection antibody that remained after the washing step, which is proportional to the amount of the polypeptide encoded by the transgene in the sample. Alternatively, for example when using purified protein, the amount or concentration of polypeptide encoded by the transgene may be determined spectrophotometrically.

For example, a suitable ELISA assay kit is the BIOPHEN FVIII:C assay (Ref: 221406) manufactured by HYPHEN BioMed as used in the Examples. If the transgene encodes a polypeptide having a Factor VIII activity, the level of the polypeptide having Factor VIII activity may be measured using the BIOPHEN FVIII:C assay.

Alternatively, the skilled person may assess the level of polypeptide encoded by the transgene by determining the activity of the expressed polypeptide encoded by the transgene.

For example, if the transgene encodes a polypeptide having a Factor VIII activity, the level of polypeptide encoded by the transgene may be determined using a chromogenic assay, such as a chromogenic assay that measures cofactor activity. For example, a suitable chromogenic assay is as follows. The polypeptide encoded by the transgene is mixed with human Factor X polypeptide and Factor IXa polypeptide, thrombin, phospholipids and calcium. The thrombin activates the polypeptide encoded by the transgene (having Factor VIII activity such as a Factor VIII polypeptide) to form Factor Villa polypeptide. The thrombin-activated polypeptide having Factor VIII activity forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess.

Therefore the limiting factor is Factor Villa polypeptide. Thus, the level of pNA generated is proportional to the amount of the Factor Xa polypeptide generated by the polypeptide having Factor VIII activity in the sample, which is proportional to the activity of polypeptide having Factor VIII activity in the sample. The activity of polypeptide having Factor VIII activity in the sample is a measure of the cofactor activity of the polypeptide having Factor VIII activity in the sample.

For example, a suitable chromogenic assay is the BIOPHEN FVIII:C assay (Ref: 221406) manufactured by HYPHEN BioMed as used in the Examples. The activity of the polypeptide having Factor VIII activity may be measured using the BIOPHEN FVIII:C assay.

The transgene used for the comparison may encode Factor VIII and in particular may encode a truncated or modified Factor VIII, such as a B-domain deleted shortened Factor VIII (“SQ”) which is well known in the art.

The present invention also provides a vector comprising a nucleotide sequence which comprises: (i) a transcription regulatory element of the invention; and (ii) a transgene as defined herein.

The vector nucleotide sequence further may further comprise a nucleotide sequence encoding a signal peptide. The nucleotide sequence encoding the signal peptide may be 50 to 100 nucleotides in length. The nucleotide sequence encoding the signal peptide may be shorter than 80 nucleotides.

The vector may be a viral particle such as an AAV vector or recombinant AAV (rAAV) vector.

The present invention also provides an AAV or rAAV vector for use in a method of treatment optionally wherein the method of treatment is a method of gene therapy. The method of treatment or gene therapy may be treatment of Haemophilia A.

The present invention also provides a method of treatment comprising administering an effective amount of an AAV or rAAV, optionally wherein the method of treatment is a method of gene therapy and/or a method of treating Haemophilia A.

The present invention also provides use of an AAV or rAAV a method of treatment, optionally wherein the method of treatment is a method of gene therapy and/or a method of treating Haemophilia A.

A “gene therapy” involves administering a vector of the invention that is capable of expressing a transgene (such as a Factor IX nucleotide sequence) in the host to which it is administered.

Optionally, the method of treatment is a method of treating a coagulopathy such as haemophilia (for example haemophilia A or B) or Von Willebrands' disease. Preferably, the coagulopathy is characterised by increased bleeding and/or reduced clotting. Optionally, the method of treatment is a method of treating haemophilia, for example haemophilia A. In some embodiments, the method of treatment comprises administering a vector of the invention to a patient. Optionally, the patient is a patient suffering from haemophilia A. Optionally, the patient has antibodies or inhibitors to Factor IX. Optionally, the vector is administered intravenously. Optionally, the vector is for administration only once (i.e. a single dose) to a patient.

When haemophilia A is “treated” in the above method, this means that one or more symptoms of haemophilia are ameliorated. It does not mean that the symptoms of haemophilia are completely remedied so that they are no longer present in the patient, although in some methods, this may be the case. The method of treatment may result in one or more of the symptoms of haemophilia A being less severe than before treatment. Optionally, relative to the situation pre-administration, the method of treatment results in an increase in the amount/concentration of circulating Factor VIII in the blood of the patient, and/or the overall level of Factor VIII activity detectable within a given volume of blood of the patient, and/or the specific activity (activity per amount of Factor IX protein) of the Factor VIII in the blood of the patient.

A “therapeutically effective amount” refers to an amount effective, at dosages and for periods of time necessary, to achieve the desired therapeutic result, such as raising the level of functional factor IX in a subject (so as to lead to functional factor VIII production at a level sufficient to ameliorate the symptoms of haemophilia B).

Optionally, the vector is administered at a dose of less than 1×10¹¹, less than 1×10¹², less than 5×10¹², less than 2×10¹², less than 1.5×10¹², less than 3×10¹², less than 1×10¹³, less than 2×10¹³, or less than 3×10¹³ vector genomes per kg of weight of patient. Optionally, the dose of vector/viral particle that is administered is selected such that the subject expresses Factor VIII at an activity of 10%-90%, 20%-80%, 30%-70%, 25%-50%, 20%-150%, 30%-140%, 40%-130%, 50%-120%, 60%-110% or 70%-100% of the Factor VIIII activity of a non-haemophilic healthy subject.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a transcription regulatory element comprising a core nucleotide sequence” should be interpreted to mean that the transcription regulatory element has at least the core nucleotide sequence, but may comprise further components such as additional nucleotide sequences.

In some embodiments of the invention, the word “comprising” is replaced with the phrase “consisting of” or the phrase “consisting essentially of”. The term “consisting of” is intended to be limiting. For example, the phrase “a core nucleotide sequence which consists of a nucleotide sequence having at least 95% identity to SEQ ID NO: 2” should be understood to mean that the core nucleotide sequence is defined with reference to SEQ ID NO: 2 and to nothing further. Similarly, the phrase “a core nucleotide sequence consisting essentially of SEQ ID NO: 2” should be understood to mean that the core nucleotide sequence comprises no additional nucleotide sequences that materially affect the function of the transcription regulatory element.

For the purpose of this invention, in order to determine the percent identity of two sequences (such as two polynucleotide sequences), the sequences are aligned for optimal comparison purposes (e.g., gaps can be introduced in a first sequence for optimal alignment with a second sequence). The nucleotides at each position are then compared. When a position in the first sequence is occupied by the same nucleotide as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, then in that instance SEQ ID NO: 1 would be the reference sequence. To assess whether a nucleotide sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

For avoidance of doubt, it will be understood that references to “at least 80% identity,” “at least 90% identity,” “at least 95% identity” and/or “at least 98% identity” should all be read as implicitly including 100% identity.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The terms “nucleic acid sequence”, “nucleotide sequence” and “polynucleotide sequence” are intended to be synonymous with one another and refer to all forms of nucleic acid, oligonucleotides, including deoxyribonucleic acid (DNA) and ribonucleic acid (RNA). Nucleic acids include naturally occurring, synthetic, and modified or altered polynucleotides. The term “nucleotide sequence” refers to a polymeric form of nucleotides of any length. The nucleotides may be deoxyribonucleotides, ribonucleotides or analogs thereof.

Where a “nucleic acid sequence”, “nucleotide sequence” or “polynucleotide sequence” is referred to as having a length, this will generally be referred to as a given number of nucleotides, e.g. 4,000 (or 4 k) nucleotides. Alternatively a nucleotide sequence length may be defined as a given number of base pairs (bp), e.g. 118 bp. It is understood that ‘base pair’ terminology generally refers to double-stranded nucleotides. However, it is not uncommon in the art to refer to a single-stranded (ss) nucleic acid by the number of base pairs it has e.g. when lined up with its complementary strand. Thus, the mere reference to length by base pairs should not be construed as limiting the nucleotide to its double-stranded form and—for the purposes of this application—the terms “4 k bp” and “4 k nucleotides” should be considered to be synonymous.

The term “between” when referencing a range of possible sequence lengths should be understood as including the end-points of that range. Thus, a reference to “between 80 and 280 nucleotides” includes a nucleotide sequence that is 80 nucleotides long and includes a nucleotide sequence that is 280 nucleotides long. Similarly, a reference to a sequence of nucleotides, e.g. “a sequence defined by nucleotides 303-335 of SEQ ID NO: 1” should be understood to include the recited nucleotides 303 and 335 within that sequence.

Nucleic acid molecules such as polynucleotides have “5′ ends” and “3′ prime ends” when in linear form. The “5′ end” is defined by its 5′ phosphate not linked to the 3′ oxygen of a pentose ring. The “3′ end” is defined by its 3′ oxygen not linked to a 5′ phosphate of a pentose ring. However, the terms “5′” and “3′” are also understood in the art to refer to relative positions within a nucleic acid molecule. Thus, for example, a particular nucleic acid, or sequence of nucleic acids, may be said to be located 5′ to, or 5′ of, a given sequence when—relative to that given sequence—the particular nucleic acid, or sequence of nucleic acids, is closer to the 5′ end than the given sequence is. In a similar manner, a particular nucleic acid, or sequence of nucleic acids, may be said to be located 3′ to, or 3′ of, a given sequence when—relative to that given sequence—the particular nucleic acid, or sequence of nucleic acids, is closer to the 3′ end than the given sequence is. Terms such as “upstream” or “downstream” are also used to describe such relative locations; within this application, the terms “5′ to”, “located 5′ to”, “5′ of”, “located 5′ of” and “upstream” are all to be regarded as synonymous. Likewise, the terms “3′ to”, “located 3′ to”, “3′ of”, “located 3′ of” and “downstream” are all to be regarded as synonymous. It will be understood that a nucleotide sequence that is stated to be 3′ or 5′ of another sequence may not be directly adjacent; that is, there may be one or more additional sequences interposed between them.

A “transcription regulatory element” refers to nucleic acid sequence(s) which influence, control or effect expression of a nucleic acid (usually a gene) to which the transcription regulatory element is operably linked. Transcription regulatory elements include promoters and enhancers. Optionally, a transcription regulatory element of the invention comprises a promoter and an enhancer. A vector sequence such as an AAV or rAAV vector sequence will generally include one or more “transcription regulatory elements” to facilitate transcription of a heterologous polynucleotide such as a transgene.

The term “operably linked” means that the transcription regulatory element of the invention is present at an appropriate position relative to another nucleic acid sequence (such as a transgene) so as to effect expression of that nucleic acid sequence.

Transcription regulatory elements of the invention may be tissue-specific, i.e. they may direct/initiate transcription in a particular cell type to a greater extent than in other tissue types. For instance the transcription regulatory element of the invention may be liver-specific.

The transcription regulatory element of the invention may be comprised within an AAV vector for use in gene therapy. A “gene therapy” involves administering AAV/viral particles capable of expressing a transgene (such as a Factor VIII-encoding nucleotide sequence) in the host to which it is administered. In such cases, the vector plasmid will generally comprise an expression cassette.

As described herein, an “expression cassette” refers to a nucleotide sequence of nucleic acids comprising a transgene and a transcription regulatory element of the invention, operably linked to the transgene. Optionally, the cassette further comprises additional transcription regulatory elements, such as enhancers, introns, untranslated regions, transcriptional terminators etc.

An expression cassette may comprise at least one ITR. The expression cassette will more typically comprise two ITRs (generally with one either end of the expression cassette, i.e. one at the 5′ end and one at the 3′ end). There may be intervening sequences between the expression cassette and one or more of the ITRs. The expression cassette may be incorporated into a viral particle located between two regular ITRs or located on either side of an ITR engineered with two D regions. Optionally, the expression cassette comprises ITR sequences which are derived from AAV1, AAV2, AAV4 and/or AAV6. Preferably the ITR sequences are AAV2 ITR sequences.

A “transgene” is used here to refer to a nucleic acid (usually heterologous) that is intended to be introduced (or which has been introduced via e.g. a vector) into a cell. A transgene may be a gene that encodes a polypeptide or protein of particular interest or it may encode e.g. a non-translated RNA which optionally is an siRNA or miRNA or a snRNA or an antisense RNA or other inhibitory nucleic acid. If the transgene is intended for expression in the liver, the transcription regulatory element may be liver-specific, i.e. it promotes substantially more protein expression in liver cells than in other tissue types. Optionally, the TRE may be a liver-specific promoter which may be a human liver-specific promoter. The transgene may be any suitable gene. If the vector plasmid is for use in gene therapy, the transgene may be any gene that comprises or encodes a protein or nucleotide sequence that can be used to treat a disease. For example, the transgene may encode an enzyme, a metabolic protein, a signalling protein, an antibody, an antibody fragment, an antibody-like protein, an antigen, or a non-translated RNA such as an miRNA, siRNA, snRNA, or antisense RNA.

### DESCRIPTION OF THE FIGURES

The present invention will now be described by way of non-limitative example with reference to the following figures, in which:

FIG. 1 shows a schematic diagram of several transcription regulatory elements of the invention (allocated internal designations FRE43, FRE49, FRE56, FRE59, FRE63 and FRE72), indicating (shaded regions) the nucleotide regions of the HLP2 TRE which are retained. Arrows indicate insertion of a fragment of the core region into the 5′ region (*) and insertion of nucleotides 265-272 of HLP2 following deletion of nucleotides 243-283 (**).

FIG. 2 shows for comparative purposes a global sequence alignment of various transcription regulatory elements derived from the HLP2 TRE. The nucleotides that form part of the consensus or core region (i.e. nucleotides 170-242 of HLP2) are indicated (*).

FIG. 3 shows a transcription regulatory element (TRE) of the invention as part of an overall Factor VIII expression cassette for use in the experiments described below. Looped Inverted Terminal Repeats (ITRs) bracket the cassette, which includes a transcription regulatory element of the invention (P), a nucleotide sequence encoding a signal peptide (SP), a nucleotide sequence encoding a truncated Factor VIII (hFVIII-SQ) and a synthetic polyA sequence (SpA). Overall length (L) of the expression cassette clearly depends on the length of the various elements.

FIG. 4 shows the results of two (4A-C i and ii) in vitro studies conducted using the exemplified transcription regulatory elements of the invention in a Factor VIII expression cassette. HuH7 cells were transfected with FVIII-SQ constructs comprising a TRE of interest. The level of FVIII activity in culture supernatant was analysed at day 3 post-transfection. 4A)i) and 4 A)ii) show the FVIII levels (% FVIII:C, determined using the FVIII chromogenic activity assay described below); 4B)i) and 4B)ii) show luciferase activity level from the corresponding transfected well; 4C)i) and 4C)ii) show the FVIII level from 4A)i) and 4A)ii) normalised to the level of luciferase expression; thereby showing the relative efficacy of the transcription regulatory element. HLP2 is provided for comparison purposes. The bar charts represent mean values from triplicate experiments. RLU=relative luminescence units.

FIG. 5 shows the results of in vivo studies conducted using the exemplified transcription regulatory elements of the invention in a Factor VIII expression cassette. 6-8-week-old male C57BL/6 mice were intravenously injected with 2×10¹² vg/kg viral vector. Six mice were injected per construct. On day 28 post injection the mice were culled and blood harvested intro citrate anticoagulant. Blood and murine liver were provided for analysis. Blood was used to carry out FVIII analysis while liver biopsies were used to calculate the vector genome. 5A)i), 5A)ii), 5A)iii) and 5A)iv) show Factor VIII antigen levels (determined using the FVIII sandwich ELISA antigen assay described below); 5B)i), 5B)ii), 5B)iii) and 5B)iv) show estimated vector genomes per liver cell; 5C)i), 5C)ii), 5C)iii) and 5C)iv) show FVIII antigen levels normalised to the vector genomes per cell thereby showing the relative efficacy of the transcription regulatory element. The bar charts represent mean values (n=6).

FIG. 6 shows the results of in vitro studies into the promoter fidelity of FRE72. FRE72 promoter fidelity was assessed in cell lines from a range of different tissues; Huh7: liver. HEK293T: kidney. PANC1: pancreas. BxPC-3: pancreas. MCF7: breast. 1643: neuroblastoma. MRC-9: lung. 697: early B cell. Cells were transduced with the control vector AAVS3.CAG.GFP or AAVS3.FRE72.GFP or untreated at MOI of 1×10⁵. FIG. 6 shows three columns for each cell type; the left hand column for each cell type (grey) relates to cells transduced with AAVS3.FRE72.GFP; the central column for each cell type (black) relates to cells transduced with the control vector; and the right hand column for each cell type (white) relates to untreated cells. For the HEK293T and MCF-7 cells the left hand (“grey”) column is so small that it is not visible in FIG. 6; similarly for the HEK293T, 1643 and 697 cells the right hand (“white”) columns is so small that it is not visible in FIG. 6.

FIG. 7 shows the results of an in vivo study conducted to confirm the longevity of the FRE72 promoter. An AAV8 construct comprising an FVIII-SQ transgene under the transcriptional control of the FRE72 promoter was prepared and administered to wild-type mice. Blood samples were taken by tail bleeding (at days 31, 56 and 104 post-injection) and finally via cardiac puncture (at day 230 post-injection). The FVIII antigen level in each sample was measured and the data points are shown on the graph. The bars represent median values.

FIG. 8 shows the results of an in vitro study comparing expression of a human protein following plasmid transfection in Huh7 cells. The plasmids used either the FRE72 promoter, or the known HCR-hAAT or HLP2 promoters. The level of protein in culture supernatant was measured at day 3 post-transfection using an ELISA. A CMV-luciferase control plasmid was used as co-transfection vector for normalisation of transfection efficiency. FIG. 8A shows the results prior to luciferase correction and FIG. 8B shows the results following luciferase correction. The bars represent median values.

### EXAMPLES

Materials and Methods

FVIII Constructs

The cDNA of human FVIII-SQ (encoding FVIII containing a 14 amino acid linker region in place of the B domain, as discussed above) was cloned into a liver-specific promoter-driven adeno-associated virus (AAV) vector.

Two different codon optimised FVIII variants (termed “co02” and “co19”) were used. To reduce the AAV recombinant genome size, a number of small liver-specific promoters were designed as set out below.

Generation of AAV vectors

AAV particles were produced by triple plasmid transfection of HEK293T cells with plasmids encoding the AAV Rep and Cap functions; adenoviral helper functions; and the recombinant genome containing the FVIII expression cassette flanked by AAV2 ITRs. Cell pellet and supernatant were harvested 72 hours post-transfection and AAV particles purified by affinity chromatography using resins such as POROS Capture Select and AVB Sepharose. AAV was then dialysed into PBS overnight, stored at 4° C. and titred by qPCR.

Assays

FVIII Chromogenic Activity Assay

The Biophen FVIII:C chromogenic assay (Hyphen BioMed, ref 221406) measures the cofactor activity of FVIII FVIII:C.

Through thrombin activation, the FVIII:C polypeptide forms a complex with human Factor IXa, phospholipids and calcium. Under these conditions, Factor X, provided in this assay at a specific concentration and in excess, is converted into Factor Xa (activated). This Factor Xa produced is directly proportional to FVIII:C, the limiting factor. Factor Xa is directly measured by a chromogenic substrate, Sxa-11. Factor Xa cleaves the chromogenic substrate and releases pNA. Production of pNA is proportional to Factor Xa activity, which is directly related to FVIII:C activity. The level of pNA released can determined by measuring colour development at 405 nm, and this is relative to the amount of the Factor Xa polypeptide generated by Factor VIII:C in the sample, which is proportional to the activity of FVIII:C in the sample.

The assay is performed according to manufacturer's instructions. Briefly, to a microplate well, preincubated at 37° C., 50 μl of calibrator plasmas, diluted (in reagent R4) test plasmas or cell supernatants/lysates or controls, is added, followed by 50 μl each of reagent R1 and R2, which are reconstituted with 6 mL of distilled water and prewarmed to 37° C. After mixing, these components form a 150 μl reaction that is allowed to incubate for 5 min at 37° C. Subsequently, the reaction is supplemented with reagent R3, which is itself resuspended in 6 mL of distilled water and prewarmed to 37° C., and the 200 μL mix is allowed to incubate for a further 5 min at 37° C. The reaction is stopped by adding 50 μl of 20% acetic acid or citric acid (20 g/l), before measuring the absorbance of the resulting 250 μl mixture at 405 nm.

Reagents:

R1—Human Factor X, lyophilized in presence of a fibrin polymerization inhibitor.

R2—Activation Reagent—Factor IXa (human), at a constant and optimized concentration, containing human thrombin, calcium and synthetic phospholipids, lyophilized.

R3—SXa-11—Chromogenic substrate, specific for Factor Xa, lyophilized, with a thrombin inhibitor.

R4—Tris-BSA Buffer. Contains 1% BSA, PEG, FVIII:C Stabilizer and sodium azide (0.9 g/L).

In relation to the readout from the chromogenic activity assay, the “% FVIII activity” (also referred to as “% FVIII:C”) is the “% normal” which means, for example in the context of expressing a FVIII expression cassette in HuH-7 cells, that relative to a human plasma sample having 100% FVIII activity, the FVIII activity detected in a supernatant following expression of a FVIII expression cassette in HuH-7 cells is a specified % of the FVIII activity detected in said human plasma sample having 100% FVIII activity.

FVIII Sandwich ELISA Antigen Assay

The Asserachrom VIII:Ag kit (Stago Diagnostica, ref 00280) is an antigenic assay for quantification of FVIII in plasma by enzyme-linked immunosorbent assay (ELISA). FVIII in assayed samples is captured by a mouse-monoclonal anti-human VIII:Ag antibody, pre-coating the walls of a plastic microplate well. Following sufficient incubation and washing to reduce non-specific binding, mouse anti-human FVIII antibodies, coupled to peroxidase, bind to the remaining free antigenic determinants of the captured FVIII. The bound peroxidase is then revealed by TMB substrate. The colour development induced by TMB is halted by the addition of a strong acid. The intensity of the colour development is directly proportional to the FVIII concentration in the assayed sample, determined by measuring the absorbance at 450 nm.

The readout from this assay may be expressed as “% normal” which means, for example in the context of expressing FVIII constructs in mice, that relative to a human plasma sample having 100% FVIII activity, the number of FVIII molecules (strictly, epitopes) detected in a mouse plasma sample is a specified % of the number of FVIII molecules/epitopes detected in said human plasma sample having 100% FVIII activity.

In both the activity and antigen assays described above, FVIII (activity or antigen levels) is quantified in mouse, or human cell supernatant, samples using the manufacturer recommended or included lyophilized human plasma samples of known FVIII activity or antigen (as appropriate), calibrated against the WHO International Standard (NIBSC code 07/316).

### Example 1—Design and Selection of Small Liver-Specific Transcription Regulatory Elements

A number of different TREs were designed based on the HLP2 TRE and selected based on overall length. Deletions to the TRE were applied, based on looking at conserved regions in the related alpha-1-antitrypsin TRE in various vertebrates. It was surprisingly found that TREs could be made which are significantly shorter than the HLP2 TRE but which retain a degree of functionality. In some cases the level of activity is at least comparable to the HLP2 TRE, as shown in the examples below.

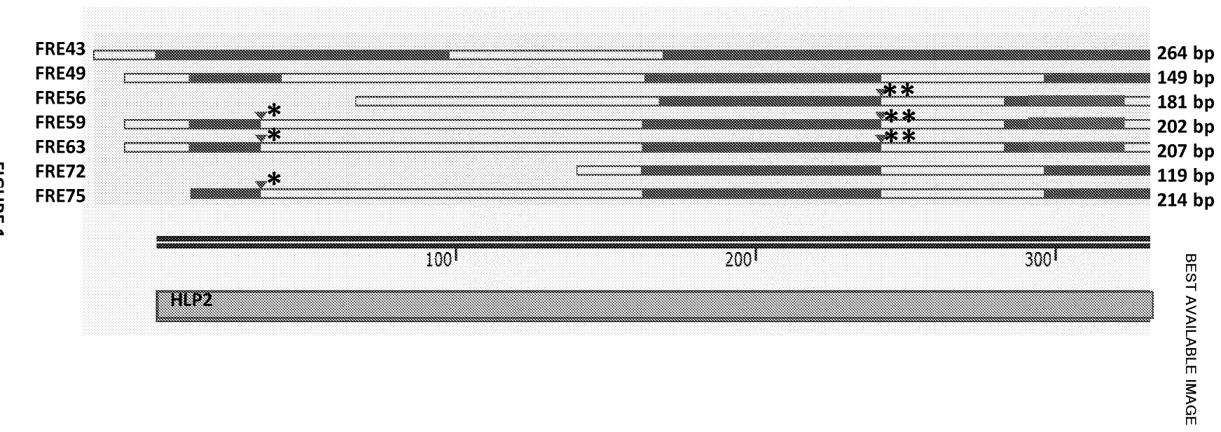

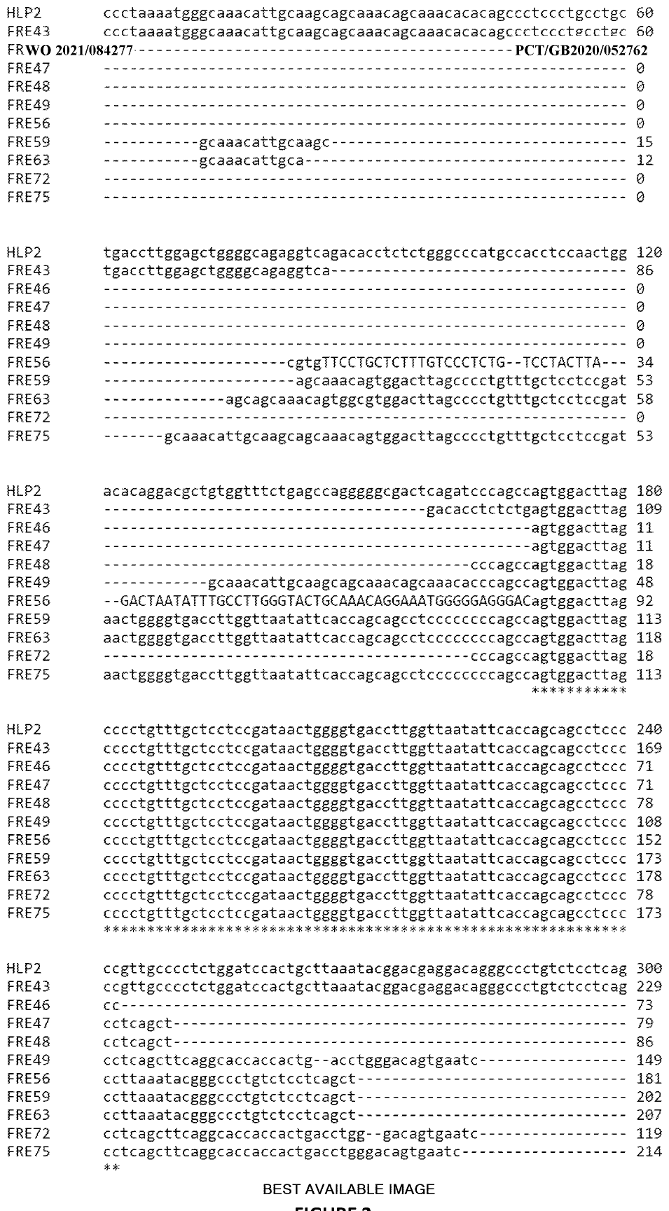

FIG. 1 shows a schematic of the HLP2 TRE and a number of transcription regulatory elements of the invention. Shaded regions represent nucleotide sequences which the present inventors have found to be highly conserved in the alpha-1-antitrypsin TRE from which HLP2 is derived. Once regions were identified for deletion, the deletion itself was carried out using known techniques. Transcription regulatory elements can be made according to the sequences disclosed herein by using known DNA synthesis techniques. Thus transcription regulatory elements of the invention can be derived from the HLP2 TRE by using deletions to remove required portions of the HLP2 sequence; or the TREs of the invention can be synthesised via site-directed mutagenesis.

The following transcription regulatory elements were designed and tested (HLP2 was used as a comparator):
FRE43 (SEQ ID NO: 24)
FRE49 (SEQ ID NO: 25)
FRE56 (SEQ ID NO: 26)
FRE59 (SEQ ID NO: 27)
FRE63 (SEQ ID NO: 28)
FRE72 (SEQ ID NO: 29)

In order to determine the minimum length required to obtain a transcription regulatory element having at least a basic level of functionality, three further transcription regulatory elements were designed and tested:
FRE46 consists of the “core nucleotide sequence” defined by SEQ ID NO: 2. FRE46 therefore corresponds to nucleotides 170-242 of SEQ ID NO:1 and is 73 nucleotides in length.
FRE47 consists of the “core nucleotide sequence” defined by SEQ ID NO: 2 together with a TSS sequence defined by SEQ ID NO: 6 located 3′ to the “core nucleotide sequence”. FRE47 therefore corresponds to nucleotides 170-242 plus nucleotides 297-302 of SEQ ID NO: 1 and is 79 nucleotides in length.
FRE48 consists of the “extended core nucleotide sequence” defined by SEQ ID NO: 3 together with a TSS sequence defined by SEQ ID NO: 6 located 3′ to the “extended core nucleotide sequence”. FRE48 therefore corresponds to nucleotides 163-242 plus nucleotides 297-302 of SEQ ID NO: 1 and is 86 nucleotides in length.

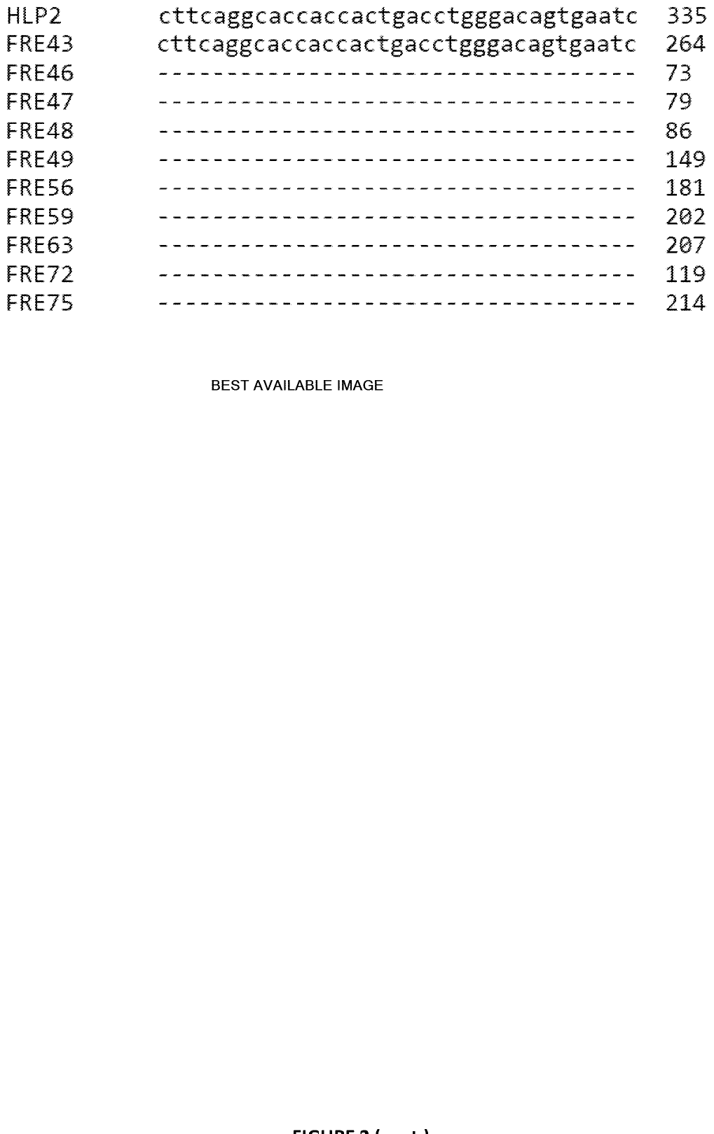

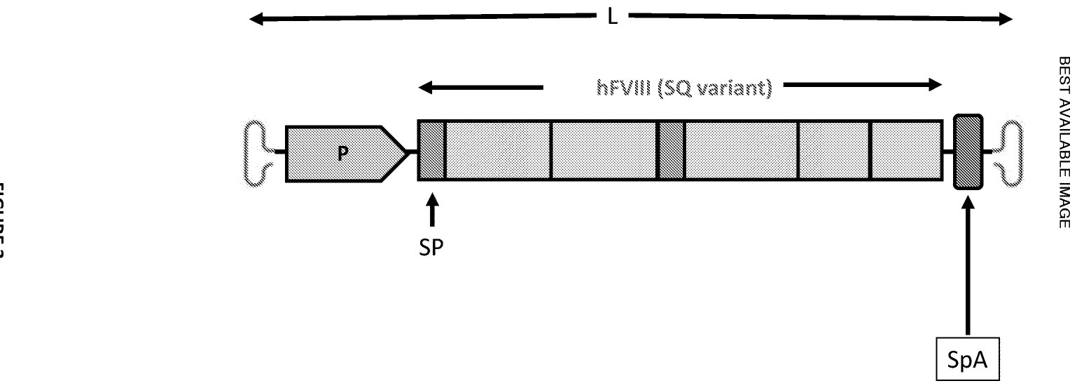

FIG. 2 shows the nucleotide sequences of the above transcription regulatory elements which were obtained using the methodology set out above. The nucleotide sequence of HLP2 is provided for comparative purposes.

### Example 2—In Vitro Assessment

To evaluate the activity of the designed transcription regulatory elements in vitro, hepatocyte derived cellular carcinoma cell line HuH7 was transiently transfected with candidate plasmids comprising the respective TREs (either HLP2 (for comparison purposes) or one of those defined in Example 1 above) positioned upstream of a designated “co02” codon-optimised transgene for a human clotting factor VIII variant (FVIII-SQ; i.e. a FVIII containing a 14 bp linker region in place of the B domain, as described e.g. in Lind et al. 1995 supra). The “co02” sequence is provided as SEQ ID NO: 31. The transgene and transcription regulatory element were flanked by ITRs from AAV2. In total 2.5×10⁵ Huh7 cells were seeded per well of a 12 well plate in DMEM low glucose+10% FBS+glutamax (D10 medium). Experiments were performed in triplicate.

For plasmid transient transfection, following FuGENE® HD Transfection protocol, 24 hours post cell seeding, 1.8 μg of plasmid (designed as set out above) and 0.2 μg CMV-Luciferase plasmids were mixed and added to FuGENE HD Reagent (8 μL). CMV-Luciferase plasmid (10% of the total plasmid) was included in each transfection in order to monitor the transfection efficiency.

Post transfection (around 18 hours later), the medium was replaced with 500 μl fresh DMEM low glucose+10% FBS+glutamax (D10 medium). 24 h later, medium was replaced by fresh DMEM low glucose+glutamax (DO media). Cells and medium were harvested the following day, i.e. at day 3 post-transfection.

FVIII activity was assessed using the BIOPHEN FVIII:C(6) (ref. 221406) kit. The absorbance was measured on SpectraMax i3. In parallel, cells were lysed (Promega E397A lysis buffer) and subjected to a Luciferase assay (Promega E1501) to measure the Luciferase expression. Luciferase expression was used as an internal control to normalise the FVIII activity. Analysis was performed using the software Graphpad Prism v7.

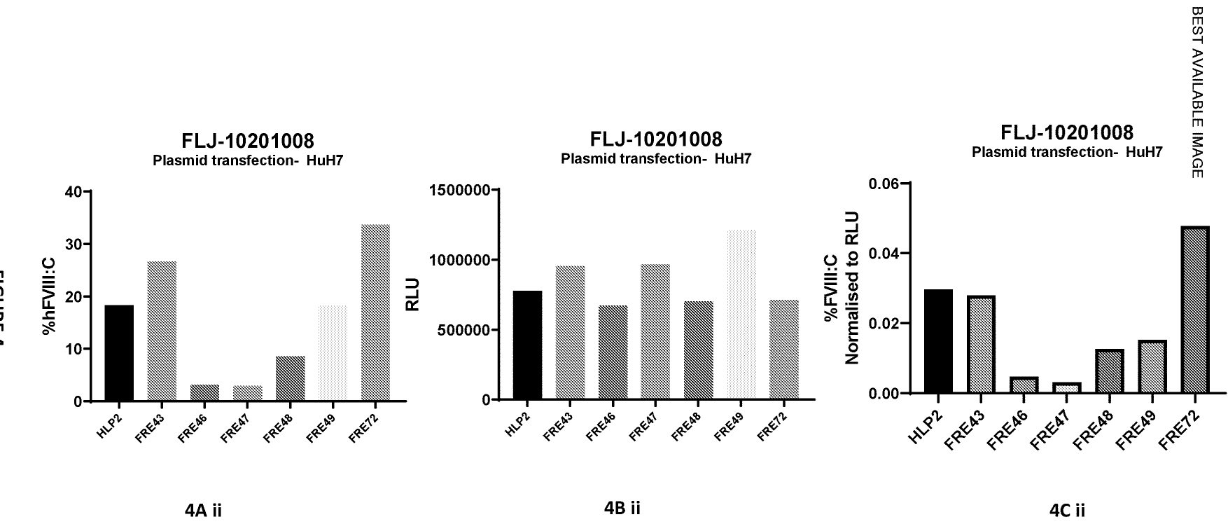

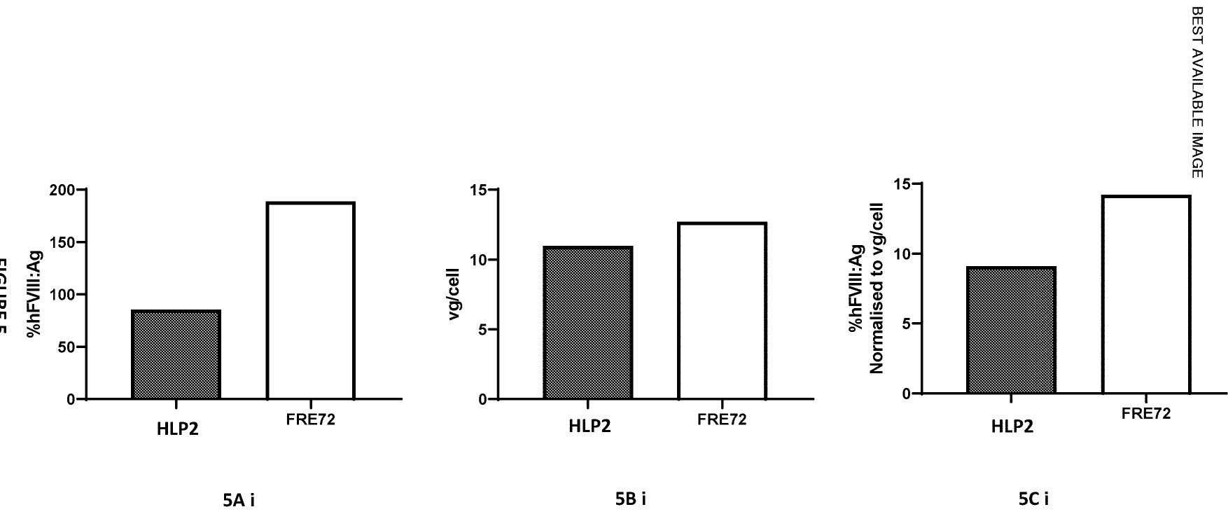

The results of the in vitro experiments are shown in FIG. 4. The most relevant panels are 4(C i) and 4(C ii), which show the relative mean FVIII expression achieved by the various TREs when normalised to the level of transfection.

### Example 3—In Vivo Assessment

AAV particles were produced as described above having a genome comprising a codon-optimised nucleotide sequence (designated “co19”) encoding a human FVIII-SQ encapsidated by an AAV8 capsid. The “co19” sequence is provided as SEQ ID NO: 32. AAV particles were produced as described above.

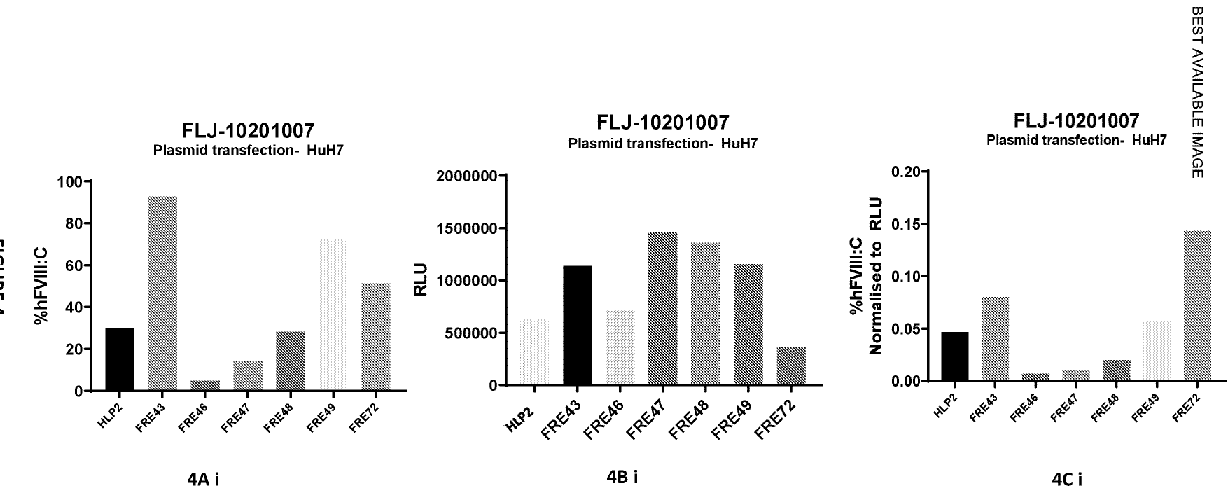

FIG. 3 provides a schematic showing the cassette obtained using the above methodology. The element P represents a transcription regulatory element such as a promoter/enhancer, which is either derived from HLP2 or is HLP2 (which was used for comparative purposes to assess utility of the TREs derived therefrom). The native FVIII signal peptide was replaced with a wild-type coding sequence for a heterologous signal peptide (termed “SP8”) which is 72 bp in length.

6-8-week-old male C57BL/6 mice were intravenously injected with 2×10¹² vg/kg viral vector. Six mice were injected per construct. On day 28 post-injection the mice were culled, and blood harvested into citrate anticoagulant. Blood and murine liver were provided for analysis.

Blood was used to carry out FVIII analysis while liver biopsies were used to calculate the vector genome.

To determine the number of vector genomes per liver cell post-AAV injection, DNA was isolated from frozen liver samples, approximately 40 mg, using QIAGEN DNeasy Blood and Tissue Kit (QIAGEN) following manufacturers' instructions. Quantitative real-time PCR (q-PCR) amplification was carried out using the PowerUp SYBR Green Master mix (Applied Biosystems) according to the manufacturer's instructions. q-PCR was performed on a QuantStudio™ instrument (Applied Biosystems). The primer sets are designed to quantify the transgene, allowing an estimation of AAV copy number. Genome copy number was calculated from the standard curve and after normalization to mouse GAPDH quantified by qPCR.

To determine the levels of FVIII protein post-AAV injection, FVIII antigen level from citrated plasma was measured by Asserachrom VIII:Ag ELISA kit (Diagnostica Stago) following manufacturer's instructions. Further dilutions were performed when deemed necessary.

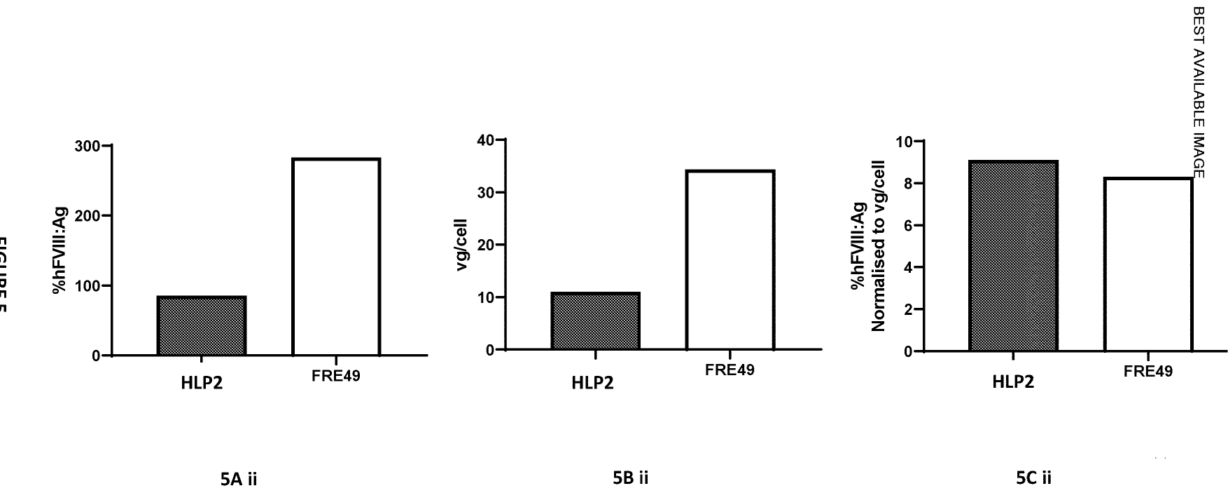

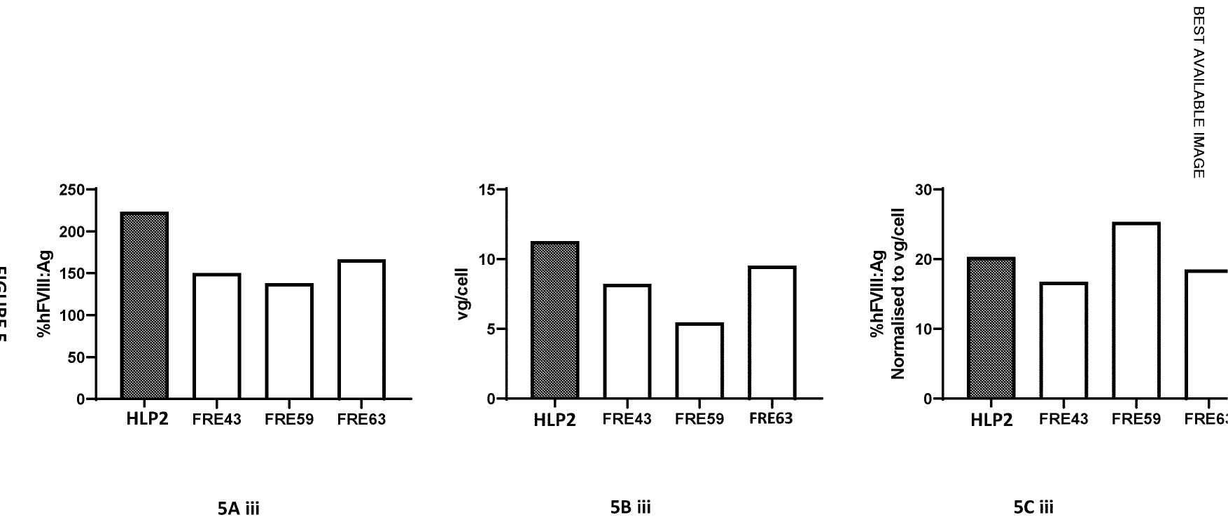

Results are shown in FIG. 5. The most relevant panels are in FIGS. 5(C)(i)-5(C)(iv), which show the relative FVIII level, normalised to the viral genome level per cell.

### Example 4—Assessing Tissue Specificity of FRE72

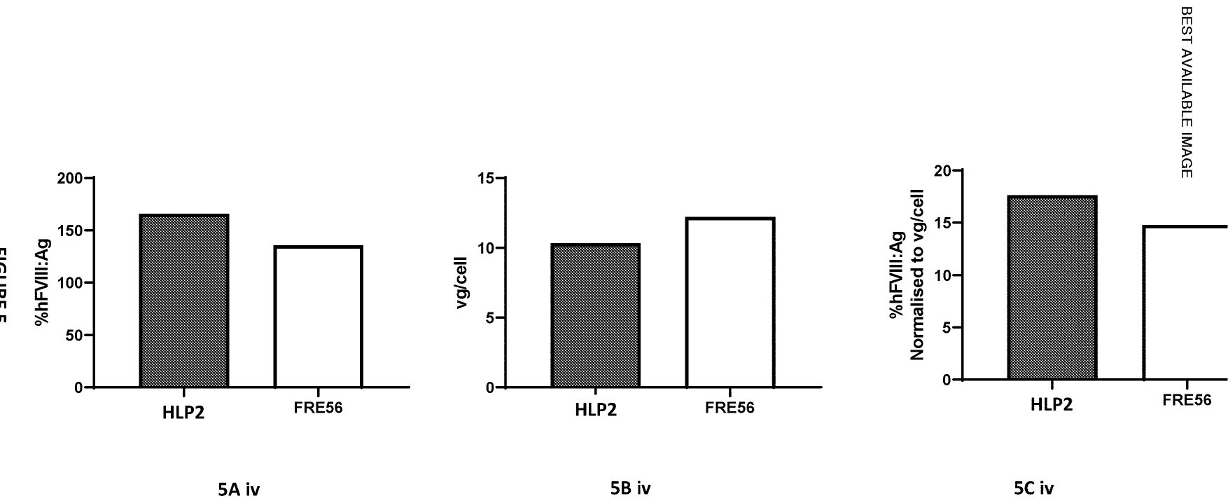

FIG. 6 shows the results of in vitro studies into the promoter fidelity of FRE72. FRE72 promoter fidelity was assessed in cell lines from a range of different tissues; Huh7: liver. HEK293T: kidney. PANC1: pancreas. BxPC-3: pancreas. MCF7: breast. 1643: neuroblastoma. MRC-9: lung. 697: early B cell. Cells were transduced with the control vector AAVS3.CAG.GFP or AAVS3.FRE72.GFP or untreated at MOI of 1×10⁵. FIG. 6 shows three columns for each cell type; the left hand column for each cell type (grey) relates to cells transduced with AAVS3.FRE72.GFP; the central column for each cell type (black) relates to cells transduced with the control vector; and the right hand column for each cell type (white) relates to untreated cells. For the HEK293T and MCF-7 cells the left hand (“grey”) column is so small that it is not visible in FIG. 6; similarly for the HEK293T, 1643 and 697 cells the right hand (“white”) columns is so small that it is not visible in FIG. 6.

### Example 5—the FRE72 Promoter Provides Long-Term Expression In Vivo

An AAV vector comprising a FVIII-SQ transgene (designated FVIIIco19-SQ) under the transcriptional control of the FRE72 promotor was pseudotyped with AAV8 capsid. The overall vector genome, including ITRs, promoter and transgene, was 4845 bp long (SEQ ID NO: 34).

The resulting AAV8 vector was administered into the tail vein of C57BL6 wild type mice at 6-8 weeks of age. Vectors were stored at 4° C. prior to injection. Original viral suspensions were diluted in sterile X-vivo 10 (Lonza, BE04-380Q) in order to obtain an adequate inoculum yielding a dose of 2×10¹² vg/kg.

At days 31, 56 and 104 post-injection, a blood sample of 100 μl was taken from the lateral tail vein of each of the mice. At day 230 post-injection, terminal bleeding was performed and maximum volume (approx. 1 ml) blood sample was taken via cardiac puncture from heavily anaesthetised animals which were culled following the blood sample. Collected blood was diluted with citrate anticoagulant (1:10 dilution) and centrifuged at 5000 rpm for 5 minutes.

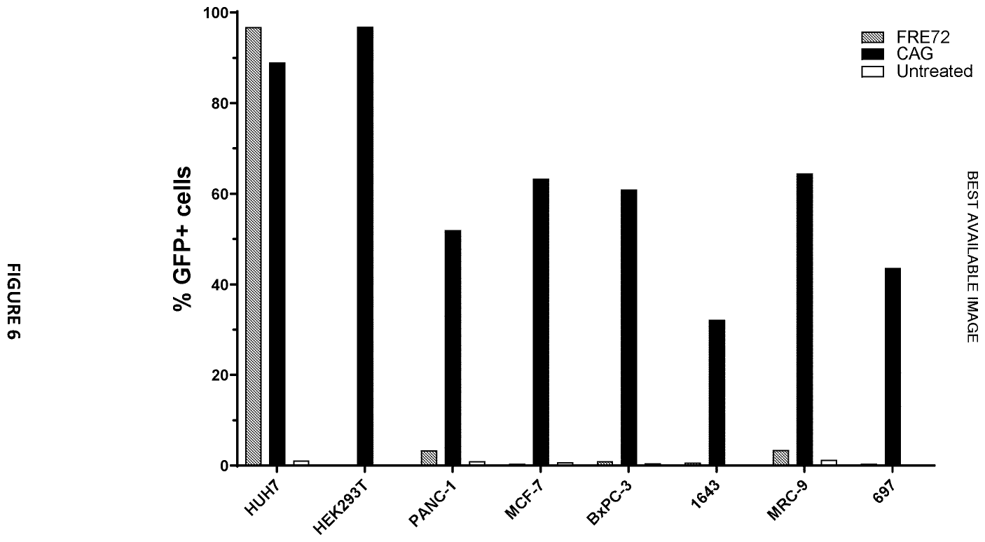

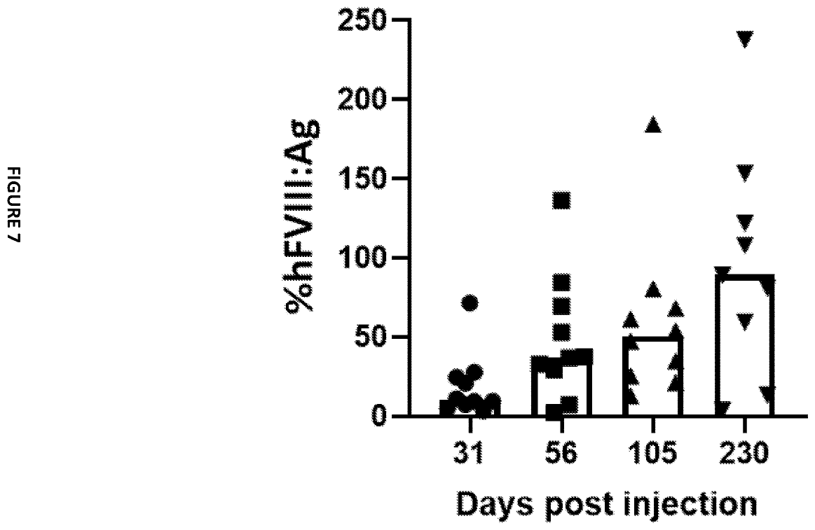

Plasma samples were analysed for FVIII antigen level using an FVIII sandwich ELISA antigen assay as described above in Materials and Methods. The results are shown in FIG. 7. The bars represent median values.

### Example 6—a Comparison of FRE72 Against the Known HLP2 and HCR-hAAT Promoters

Three separate test plasmid DNA constructs were prepared which incorporated a codon-optimised transgene sequence encoding a human protein under the control of either the FRE72 promoter or the known HLP2 or HCR-hAAT promoters.

To compare expression levels for each of the promoters, Huh7 cells (JCRB cell bank, no. JCRB0403) were seeded in a 96 well plate (30,000 cells per well) in DMEM low glucose, 10% FBS+Glutamax (D10 media) and cultured at 37° C. and 5% CO₂ (day 1). The next day (approx. 24 hours after cell seeding; day 2), the plasmid DNA-transfection reagent mixture was prepared and transfected into Huh7 cells. 0.225 μg of test plasmid DNA and 0.025 μg CMV-Luciferase control plasmid (FLJ-PL282) were mixed with FuGENE at a ratio of 4 μl FuGENE per μg of DNA (or 1 μl FuGENE per 0.25 μg of plasmid DNA). For 96-well transfection experiments, 1 μl of the FuGENE mix was added per well. The plasmid DNA-transfection reagent mixture was incubated on the cells overnight at 37° C. and 5% CO₂.

The next morning (day 3), approximately 18 hours after transfection, the media was replaced with fresh D10 media and cells incubated overnight at 37° C. and 5% CO₂. The next morning (24 h later; day 4), media was replaced by fresh DMEM low glucose+Glutamax+Insulin-Transferrin-Selenium supplement (DO/ITS media). Cells and media were harvested the following day (on day 5).

Protein expression in culture media was assessed using an ELISA kit. In parallel to the ELISA, Huh7 cells were washed with Phosphate buffered saline (PBS) twice and cells treated with 100 μl of luciferase lysis buffer of the Luciferase assay kit (Promega cat# E1501/E4530). Cell lysates were stored at −80° C. On the day of luciferase assays, cell lysates were thawed and 20 μl of the sample was used to measure the luciferase expression by luminescence on a Molecular Devices SpectraMax i3x plate reader. The detailed protocol is published in the Promega Technical Bullitin #TB281. Luciferase expression was used as internal control to normalise the protein levels. Analysis were performed using the software Graphpad Prism v7.

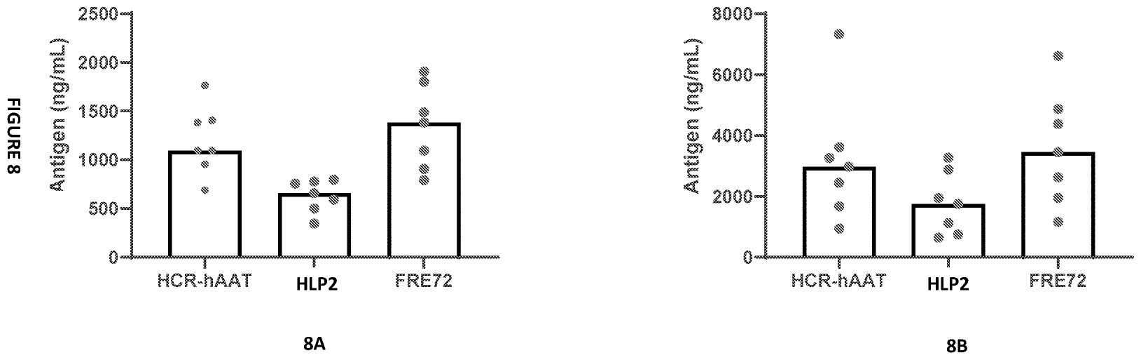

The results are shown in FIG. 8A (before luciferase correction) and FIG. 8B (after luciferase correction).

Sequence listing table

| SEQ ID NO. | Sequence description/derivation |
|---|---|
| 1 | Known HLP2 TRE as disclosed e.g. in WO16/181122 |
| 2 | The “core nucleotide sequence” i.e. a nucleotide sequence from the |
| | HLP2 TRE which the present inventors have discovered is common to |
| | all TREs of the invention. Corresponds to nucleotides 170-242 of SEQ |
| | ID NO: 1. |
| 3 | An extended “core nucleotide sequence” which is common to a |
| | subset of TREs of the invention. Corresponds to nucleotides 163-242 |
| | of SEQ ID NO: 1. |
| 4 | A part of the HLP2 TRE (located 5′ to the “core nucleotide sequence”) |
| | which the present inventors have discovered can be deleted, |
| | truncated or modified while retaining functionality. Corresponds to |
| | nucleotides 118-162 of SEQ ID NO: 1. |
| 5 | A part of the HLP2 TRE (located 3′ to the “core nucleotide sequence”) |
| | which the present inventors have discovered can be deleted, |
| | truncated or modified while retaining functionality. Corresponds to |
| | nucleotides 243-283 of SEQ ID NO: 1. |
| 6 | A transcription start site (“TSS”) found in the HLP2 TRE (located 3′ to |
| | the “core nucleotide sequence”). Corresponds to nucleotides 297-302 |
| | of SEQ ID NO: 1. |
| 7 | A transcription start site (“TSS”) found in the HLP2 TRE (located 3′ to |
| | the “core nucleotide sequence”). Corresponds to nucleotides 303-308 |
| | of SEQ ID NO: 1. |
| 8 | A transcription start site (“TSS”) found in the HLP2 TRE (located 3′ to |
| | the “core nucleotide sequence”). Corresponds to nucleotides 314-319 |
| | of SEQ ID NO: 1. |
| 9 | A part of the HLP2 TRE (located 3′ to the “core nucleotide sequence”) |
| | which the present inventors have discovered can be retained in some |
| | TREs of the invention. Corresponds to nucleotides 284-302 of SEQ ID |
| | NO: 1. |
| 10 | A part of the HLP2 TRE (located 3′ to the “core nucleotide sequence”) |
| | which the present inventors have discovered can be retained in some |
| | TREs of the invention. Corresponds to nucleotides 297-335 of SEQ ID |
| | NO: 1. |
| 11 | A part of the HLP2 TRE (located 3′ to the “core nucleotide sequence”) |
| | which the present inventors have discovered can be retained in some |
| | TREs of the invention. Corresponds to nucleotides 265-272 of SEQ ID |
| | NO: 1. May be considered to be a truncation of SEQ ID NO: 5 (q.v.). |
| 12 | Part of an exemplified TRE of the invention (designated ″FRE43″) |
| | located 3' to the “core nucleotide sequence”. |
| 13 | Part of several exemplified TREs of the invention (designated |
| | “FRE56”, “FRE59” and “FRE63”) located 3′ to the “core nucleotide |
| | sequence”. |
| 14 | A part of the HLP2 TRE (located 5′ to the “core nucleotide sequence”) |
| | which the present inventors have discovered can be retained in some |
| | TREs of the invention. Corresponds to nucleotides 12-33 of SEQ ID NO: |
| | 1. |
| 15 | A part of the HLP2 TRE (located 5′ to the “core nucleotide sequence”) |
| | which the present inventors have discovered can be retained in some |
| | TREs of the invention. Corresponds to nucleotides 12-41 of SEQ ID NO: |
| | 1. |
| 16 | A part of the HLP2 TRE (located 5′ to the “core nucleotide sequence”) |
| | which the present inventors have discovered can be retained in some |
| | TREs of the invention. Corresponds to nucleotides 1-98 of SEQ ID NO: |
| | 1. |
| 17 | A part of the HLP2 TRE (located 5′ to the “core nucleotide sequence”) |
| | which the present inventors have discovered can be retained in some |
| | TREs of the invention. Corresponds to nucleotides 163-169 of SEQ ID |
| | NO: 1. Comprised within the “extended core nucleotide sequence” |
| | defined by SEQ. ID NO: 3 (q.v.). |
| 18 | Part of an exemplified TRE of the invention (designated “FRE43”) |
| | located 5′ to the “core nucleotide sequence”. |
| 19 | Part of an exemplified TRE of the invention (designated “FRE49”) |
| | located 5′ to the “core nucleotide sequence”. |
| 20 | Part of an exemplified TRE of the invention (designated “FRE56”) |
| | located 5′ to the “core nucleotide sequence”. |
| 21 | Part of an exemplified TRE of the invention (designated “FRE59”) |
| | located 5′ to the “extended core nucleotide sequence”. |
| 22 | Part of an exemplified TRE of the invention (designated “FRE63”) |
| | located 5′ to the “core nucleotide sequence”. |
| 23 | Part of an exemplified TRE of the invention (designated “FRE72”) |
| | located 5′ to the “core nucleotide sequence”. |
| 24 | An exemplified TRE of the invention (designated “FRE43”). |
| 25 | An exemplified TRE of the invention (designated “FRE49”). |
| 26 | An exemplified TRE of the invention (designated “FRE56”). |
| 27 | An exemplified TRE of the invention (designated “FRE59”). |
| 28 | An exemplified TRE of the invention (designated “FRE63”). |
| 29 | An exemplified TRE of the invention (designated “FRE72”). |
| 30 | A possible enhancer region located 5′ to the “core nucleotide |
| | sequence” |
| 31 | A codon-optimised nucleotide sequence (designated “co02”) which |
| | encodes a FVIII-SQ peptide |
| 32 | A codon-optimised nucleotide sequence (designated “col9”) which |
| | encodes a FVIIl-SQ peptide |
| 33 | Known HCR-hAAT TRE |
| 34 | An AAV vector genome as used in Example 5 incorporating the FRE72 |
| | promoter with a FVIII-SQ transgene (designated FVIIIco19-SQ) |
| 35 | FRE75TRE |
| 36 | FRE46TRE |
| 37 | FRE47TRE |

SEQUENCES
- HLP2 TRE
>SEQ ID NO: 1
ccctaaaatgggcaaacattgcaagcagcaaacagcaaacacacagccctccctgcctgctgaccttggagct
ggggcagaggtcagacacctctctgggcccatgccacctccaactggacacaggacgctgtggtttctgagcc
agggggcgactcagatcccagccagtggacttagcccctgtttgctcctccgataactggggtgaccttggtt
aatattcaccagcagcctcccccgttgcccctctggatccactgcttaaatacggacgaggacagggc
cctgtctcctcagcttcaggcaccaccactgacctgggacagtgaatc
- Core nucleotide sequence
>SEQ ID NO: 2
agtggacttagcccctgtttgctcctccgataactggggtgaccttggttaatattcaccagcagcctccccc
- Extended core nucleotide sequence
>SEQ ID NO: 3
cccagccagtggacttagcccctgtttgctcctccgataactggggtgaccttggttaatattcaccagcagc
ctccccc
- 5′ section of HLP2 (118-162 of SEQ ID 1)
>SEQ ID NO: 4
tggacacaggacgctgtggtttctgagccagggggcgactcagat
- 3′ section of HLP2 (243-283 of SEQ ID 1)
> >SEQ ID NO: 5
gttgcccctctggatccactgcttaaatacggacgaggaca
- TSS
> >SEQ ID NO: 6
tcagct
- TSS
> >SEQ ID NO: 7
tcaggc
- TSS
>SEQ ID NO: 8
cactga
- 3′ section (284-302 of SEQ ID NO: 1)
>SEQ ID NO: 9
gggccctgtc tcctcagct
- 3′ section of FRE49, FRE72 and FRE75 (297-335 of SEQ ID NO: 1)
>SEQ ID NO: 10
tcagcttcaggcaccaccactgacctgggacagtgaatc
- 3′ section 265-272 of SEQ ID NO: 1
>SEQ ID NO: 11
ttaaatac
- 3′ section of FRE43
>SEQ ID NO: 12
gttgcccctctggatccactgcttaaatacggacgaggacagggccctgtctcctcagcttcaggcaccacca
ctgacctgggacagtgaatc
- 3′ section of FRE56, FRE59 and FRE63
>SEQ ID NO: 13
ttaaatacgg gccctgtctc ctcagct
- HLP2 5′ section (12-33 of SEQ ID NO: 1)
>SEQ ID NO: 14
gcaaacattg caagcagcaa ac
- HLP2 5′ section (12-41 of SEQ ID NO: 1)
>SEQ ID NO: 15
gcaaacattg caagcagcaa acagcaaaca
- HLP2 5′ section (1-98 of SEQ ID NO: 1)
>SEQ ID NO: 16
ccctaaaatgggcaaacattgcaagcagcaaacagcaaacacacagccctccctgcctgctgaccttggagct
ggggcagaggtcagacacctctctg
- HLP2 5′ section (163-169 of SEQ ID NO: 1)
>SEQ ID NO: 17
cccagcc
- FRE43 5′ section
>SEQ ID NO: 18
ccctaaaatgggcaaacattgcaagcagcaaacagcaaacacacagccctccctgcctgctgaccttggagct
ggggcagaggtcagacacctctctg
- FRE49 5′ section
>SEQ ID NO: 19
gcaaacattgcaagcagcaaacagcaaaca
- FRE56 5′ section
>SEQ ID NO: 20
cgtgttcctgctctttgtccctctgtcctacttagactaatatttgccttgggtactgcaaacaggaaatggg
ggagggac
- FRE59 5′ section
>SEQ ID NO: 21
gcaaacattgcaagcagcaaacagtggacttagcccctgtttgctcctccgataactggggtgaccttggtta
atattcaccagcagcctccccc
- FRE63 5′ section
>SEQ ID NO: 22
gcaaacattgcaagcagcaaacagtggcgtggacttagcccctgtttgctcctccgataactggggtgacctt
ggttaatattcaccagcagcctccccc
- FRE72 5′ section
>SEQ ID NO: 23
cccagcc
- FRE43 TRE
>SEQ ID NO: 24
ccctaaaatgggcaaacattgcaagcagcaaacagcaaacacacagccctccctgcctgctgaccttggagct
ggggcagaggtcagacacctctctgagtggacttagcccctgtttgctcctccgataactggggtgaccttgg
ttaatattcaccagcagcctcccccgttgcccctctggatccactgcttaaatacggacgaggacagggccct
gtctcctcagcttcaggcaccaccactgacctgggacagtgaatc
- FRE49 TRE
>SEQ ID NO: 25
gcaaacattgcaagcagcaaacagcaaacacccagccagtggacttagcccctgtttgctcctccgataactg
gggtgaccttggttaatattcaccagcagcctccccctcagcttcaggcaccaccactgacctgggacagtga
atc
- FRE56 TRE
>SEQ ID NO: 26
cgtgttcctgctctttgtccctctgtcctacttagactaatatttgccttgggtactgcaaacaggaaatggg
ggagggacagtggacttagcccctgtttgctcctccgataactggggtgaccttggttaatattcaccagcag
cctcccccttaaatacgggccctgtctcctcagct
- FRE59 TRE
>SEQ ID NO: 27
gcaaacattgcaagcagcaaacagtggacttagcccctgtttgctcctccgataactggggtgaccttggtta
atattcaccagcagcctccccccccagccagtggacttagcccctgtttgctcctccgataactggggtgacc
ttggttaatattcaccagcagcctcccccttaaatacgggccctgtctcctcagct
- FRE63 TRE
>SEQ ID NO: 28
gcaaacattgcaagcagcaaacagtggcgtggacttagcccctgtttgctcctccgataactggggtgacctt
ggttaatattcaccagcagcctccccccccagccagtggacttagcccctgtttgctcctccgataactgggg
tgaccttggttaatattcaccagcagcctcccccttaaatacgggccctgtctcctcagct
- FRE72 TRE
>SEQ ID NO: 29
cccagccagtggacttagcccctgtttgctcctccgataactggggtgaccttggttaatattcaccagcagc
ctccccctcagcttcaggcaccaccactgacctgggacagtgaatc
- apoE enhancer, HNF5
>SEQ ID NO: 30
gcaaaca
- co02 codop FVIII-SQ
>SEQ ID NO: 31
atgcagattgagctgtctacctgcttctttctgtgcctgctgagattctgctttagtgctacaaggcgttact
atctgggagctgtggagctgtcttgggattacatgcagtcagacctgggagagctgccagtggatgccagatt
tccccctcgagtgcccaagagcttcccttttaatacctctgtggtgtataagaaaaccctgtttgtggagttt
accgatcacctgttcaacattgctaagccaaggccaccctggatgggcctgctgggaccaacaatccaggctg
aggtgtatgatacagtggtcatcaccctgaagaacatggcttcccaccctgtgtcactgcatgctgtgggagt
gagctactggaaggccagtgagggagctgagtatgatgatcagaccagccagagagagaaggaggatgacaag
gtgtttcctggaggctctcatacctatgtgtggcaggtgctgaaggagaatggcccaatggctagtgatcccc
tgtgcctgacctacagctatctgtctcatgtggacctggtgaaggatctgaacagtggcctgattggagccct
gcttgtgtgtcgtgaaggctctctggccaaggaaaagacccagacactgcataagttcatcctgctttttgct
gtgtttgatgagggcaagtcctggcacagtgagacaaagaactccctgatgcaggacagggatgctgccagtg
ccagggcctggcccaagatgcatacagtgaatggctatgtgaataggtccctgcctggcctgattggatgtca
cagaaagagtgtgtattggcatgtgattggcatgggcaccacacctgaggttcactccatcttcctggagggc
catacctttcttgtgagaaaccacaggcaggccagtctggagatcagtcctatcaccttcctgacagcccaga
ccctgcttatggatctgggccagttcctgcttttttgccacatctccagtcaccagcatgatggcatggaggc
ttatgtgaaggtggactcctgtcctgaggaacctcagctgagaatgaagaacaatgaggaagctgaggactat
gatgatgacctgacagactctgagatggatgtggttagatttgatgatgacaactctccttcctttattcaaa
tccgatcagtggccaagaaacacccaaagacatgggtgcattacattgctgcagaggaggaggactgggatta
tgctcctctggtgctggcccctgatgacaggtcctacaagtcccagtatctgaacaatggccctcagaggatt
ggcagaaagtacaagaaagtgaggttcatggcttatacagatgagacattcaagacaagggaggccatccagc
atgagagtggcatcctgggaccactgctttatggagaagtgggagacaccctgcttatcatttttaaaaacca
ggcttccaggccctacaatatctatcctcatggcatcacggatgtgagacccctgtacagtaggagactgcct
aagggagtgaagcacctgaaggacttcccaatcctgcctggagagattttcaagtataagtggacagtgacag
tggaggatggcccaaccaagagtgaccccaggtgcctgacaagatactattcttcctttgtgaatatggagag
ggacctggcctctggcctgattggacctctgcttatctgttacaaggagtctgtggatcagagaggcaaccag
atcatgagtgacaagaggaatgtgatcctgttcagtgtgtttgatgagaacaggtcttggtatctgacagaga
acatccagagattcctgcccaatcctgctggagtgcaactggaggaccctgagtttcaggcctccaacatcat
gcatagcatcaatggctatgtgtttgactccctccaactgagtgtgtgcctgcatgaggtggcttattggtac
attctgagcattggagcccagacagatttcctgagtgtgttctttagtggctacaccttcaagcataagatgg
tgtatgaggacaccctgacactgttccccttttctggagagacagtgttcatgtccatggagaatcctggcct
gtggattctgggctgccacaactctgatttccgtaatcgtggcatgacagcccttctgaaggtgtcttcctgt
gacaagaacacaggagactactatgaggattcttatgaggacatcagtgcttatctgcttagcaagaacaatg
ccattgagccaaggagcttttctcagaatcctccagtgctgaagagacaccagagagagatcacgcgtaccac
actccagagtgatcaggaggaaattgactatgatgacacaatcagtgtggagatgaaaaaggaggactttgac
atctatgatgaggatgagaaccagagccccaggtctttccagaagaaaaccagacattactttattgctgcag
tggagagactgtgggattatggcatgtccagctctccacatgtgctgagaaatagagcccagagtggcagtgt
gccccagttcaagaaagtggttttccaggagtttacagatggatcatttacacagcctctgtacagaggagag
ctgaatgagcatctgggcctgcttggcccatatatcagagctgaggtggaggataacatcatggtgaccttcc
gtaatcaggccagcaggccctactccttttattcatccctgatctcctatgaggaagaccagagacagggagc
tgagccaagaaagaactttgtgaagcccaatgagacaaagacctacttttggaaggtgcagcaccatatggcc
cctaccaaggatgagtttgattgcaaggcttgggcttacttcagtgatgtggatctggagaaggatgtgcatt
ctggcctgattggaccactgcttgtgtgccataccaacacactgaatcctgctcatggcagacaagtgacagt
gcaggagtttgccctgttctttaccatctttgatgagacaaagagctggtacttcacagagaacatggagagg
aattgcagggctccttgtaacatccagatggaggacccaaccttcaaggagaactacagatttcatgctatca
atggctatatcatggatacactgcctggcctggtcatggctcaggaccagaggatcaggtggtatctgcttag
catgggctccaatgagaatatccacagcatccatttctctggccatgtgtttaccgtgagaaaaaaggaggaa
tataagatggccctgtacaacctgtatcctggagtgtttgagacagtggagatgctgccatctaaggctggca
tctggagggtggagtgcctgattggagagcacctgcatgctggcatgtctaccctgtttctggtgtactccaa
taagtgtcagacaccactgggcatggccagtggccatatcagagatttccagatcacagcctctggacagtat
ggacagtgggctccaaagctggctagactgcactattctggctccatcaatgcctggtccaccaaggagccct
tctcctggatcaaggtggacctgcttgctcccatgatcattcatggcatcaagacacagggagccaggcagaa
gttctcttccctgtacatcagccagtttatcatcatgtattctctggatggcaagaaatggcagacctacaga
ggcaattctacaggcacactgatggtgttctttggcaatgtggacagctctggcatcaagcacaacatcttca
atccccctatcattgctagatacatcagactgcaccctacccattattctatccgatccacactgagaatgga
gctgatgggctgtgatctgaacagctgttctatgccactgggcatggagtccaaggccatcagtgatgctcag
atcacagcctccagctacttcaccaatatgtttgctacatggtcccctagcaaggccaggctgcacctccagg
gcagatccaatgcttggagacctcaagttaacaatccaaaggagtggctccaggtggattttcagaaaaccat
gaaggtgacaggagtgaccacccagggagtgaagtctctgcttaccagcatgtatgtgaaggagttcctgatc
tcttcgagtcaagatggacaccagtggacactgttctttcagaatggcaaggtgaaggtgttccagggcaatc
aggattcctttaccccagtggtgaacagcctggacccaccactgcttacaagatacctgagaatccaccctca
gtcctgggtgcatcagattgctctgaggatggaggtgctgggatgtgaggctcaggacctgtattga
- co19 codop FVIII-SQ
>SEQ ID NO: 32
atgcagattgagctctccacctgcttcttcctctgcctcttgagattctgtttctctgctactagaagatatt
atcttggggcagtggagctgagctgggactacatgcagtctgacctgggagaactgcctgtggatgccagatt
tccccctcgagtgcccaagagcttcccctttaacacctcagtggtgtacaagaagaccctgtttgtggagttt
acagaccatctcttcaacattgctaagcccagacctccctggatgggcctgctgggccctaccatccaagctg
aagtgtatgacactgttgtgatcacactcaagaacatggcctcccatcctgtgtccctgcatgcagtgggagt
ctcctactggaaggcctcagaaggagcagagtatgatgaccagaccagccagagagagaaggaggatgacaag
gtgtttcctggagggagccacacctatgtgtggcaggtgctgaaggagaatggacctatggccagtgaccctc
tgtgtcttacctattcctacctgtcacatgtggatctggtgaaggacctgaacagtggcctgattggggctct
gctggtttgcagagaaggcagcttggccaaggagaagacccaaaccctgcacaagttcatcctgctgtttgct
gtgtttgatgaggggaaatcatggcactcagagaccaagaacagcctcatgcaggatagggatgctgccagtg
ccagggcttggcccaagatgcacactgtgaatggctatgtgaatagaagcctgcctgggctgataggctgtca
cagaaaatctgtgtactggcatgtgattggcatgggcaccacacctgaggtgcactccattttcctggagggc
cacaccttccttgtgagaaaccacagacaagcttccctggagatcagcccaatcacctttctgactgctcaaa
ccctcctgatggatctgggccagttcctgctgttctgtcatatctcctcacaccagcatgatggaatggaagc
ttatgtcaaggtggactcctgcccagaggaaccacagctcagaatgaagaacaatgaggaggctgaggactat
gatgatgacctgacagactctgaaatggatgtggtcagatttgatgatgacaacagcccttcattcatccaaa
tcagatctgtggccaagaagcatcccaagacctgggtgcactacatagctgctgaggaggaggactgggacta
tgcccctctggtcctggcccctgatgacagaagctataaaagccagtacctgaataatggcccccagagaatt
ggcagaaagtacaagaaagtcagattcatggcttacactgatgagaccttcaaaaccagggaagccatccagc
atgagtcaggcatcctgggccccctgctgtatggggaggttggagataccctgctgattatcttcaaaaacca
ggcaagcaggccctacaatatctaccctcatggcatcactgatgtcaggccactgtattccagaagactgcct
aagggggtgaagcacctgaaggacttcccaatcctgccaggggagattttcaaatacaagtggacagtgactg
tggaggatggaccaaccaagtcagatcctagatgtctgaccagatactactccagctttgtgaacatggagag
agacctggcctctggcctgattggccctctgctgatctgctataaagagtcagtggaccagagaggcaaccag
atcatgagtgacaaaagaaatgtgatcttgttctcagtgtttgatgagaatagatcttggtacctcacagaaa
acatccagaggttcctgcccaatccagctggggtgcagctggaagatccagaattccaggccagcaacatcat
gcatagcatcaatggttatgtctttgacagcctgcagctgtcagtgtgtctgcatgaagttgcttactggtat
attctgtccattggagcccagacagacttcctgtctgtcttcttctctggctacacctttaaacacaagatgg
tgtatgaggacaccctgaccctgttccctttctctggggaaacagtgttcatgtccatggaaaaccctggact
gtggatcctgggctgccataacagtgacttcagaaacagaggcatgacagccctgctcaaggtgtccagctgt
gataagaacacaggagactactatgaggatagctatgaggacatcagtgcttacctgctgagcaagaataatg
ccattgaacccaggtcattttcccaaaatccccctgtgctgaaaaggcaccagagggagatcacgcgtaccac
cctgcagagtgaccaggaggaaattgattatgatgacaccatctctgtggaaatgaaaaaggaggattttgac
atctatgatgaggatgagaaccagagccctagaagcttccagaaaaagactagacactacttcattgctgcag
tggagagactctgggattatggcatgagctccagcccccatgtgctgagaaatagagctcagagtggcagtgt
gccacagttcaagaaggtggtgtttcaggagttcactgatggctccttcacacaaccactttacagaggagaa
ctgaatgagcacctgggcctcctgggcccctacatcagggctgaagtggaggataacattatggtcacattta
ggaatcaggcttccagaccctactccttttattcctcactcatttcctatgaggaggaccagaggcagggagc
tgagcccagaaaaaattttgtgaaacccaatgaaaccaagacctacttctggaaggtgcagcaccatatggcc
cctaccaaggatgaatttgactgcaaggcttgggcttacttttctgatgtggaccttgagaaagatgtgcatt
caggcctcattgggccactgctggtgtgccacaccaatacactgaaccctgctcatgggagacaggtcacagt
gcaggagtttgcactcttctttaccatctttgatgagaccaagtcctggtatttcactgagaacatggagagg
aactgcagggccccttgtaacatccagatggaggatcccaccttcaaggaaaactacagattccatgccatca
atggctacatcatggacaccctgccaggcctggtgatggcccaggaccagaggatcaggtggtacctcctgtc
tatgggcagcaatgaaaatatccacagcattcacttctctggacatgtgtttactgtgaggaagaaggaggaa
tacaagatggctctgtacaacctctaccctggggtgtttgaaacagtggagatgctgccctccaaggctggca
tctggagagtggaatgtctgattggggagcatctgcatgctggcatgagcacactgttcctggtgtattccaa
caagtgccagaccccactgggcatggcctcaggacatatcagggacttccagatcactgctagtggacaatat
ggacagtgggcacccaagctggccagactgcactactcaggctccatcaatgcctggagtaccaaggagccct
tcagctggatcaaggtggacctgctggcccccatgattatacatggcatcaagacccagggagctagacagaa
gttcagctccctgtacatctcccaattcatcatcatgtactctctggatggcaagaaatggcagacctacaga
ggcaatagcactggcaccctgatggtgttttttggaaatgttgactcttctggcatcaagcacaacatcttca
acccccccatcattgccagatatatcaggctccaccccacccactactccataaggagcaccctgagaatgga
gctgatgggctgtgacctgaattcctgctccatgcccctgggcatggaatccaaggcaatctctgatgcacag
atcacagcctcctcctacttcaccaacatgtttgcaacctggagcccctccaaggccagactgcacctgcagg
gcaggtccaatgcttggagaccacaagtgaacaacccaaaggagtggctgcaggtggacttccagaagaccat
gaaagtgactggagtgaccacccagggagtgaaatccctgctcactagcatgtatgtgaaggaattcctgatc
agtagctctcaagatggccaccagtggaccctgttcttccagaatggcaaggtgaaggtgtttcagggcaacc
aggattccttcacccctgtggtgaatagcctggatcccccactgctgaccagatacctgagaatccaccccca
gtcctgggttcaccagattgccctgagaatggaggtgctgggctgtgaggcccaggacctgtactga
- known hAAT TRE
>SEQ ID NO: 33
aggctcagaggcacacaggagtttctgggctcaccctgcccccttccaacccctcagttcccatcctccagca
gctgtttgtgtgctgcctctgaagtccacactgaacaaacttcagcctactcatgtccctaaaatgggcaaac
attgcaagcagcaaacagcaaacacacagccctccctgcctgctgaccttggagctggggcagaggtcagaga
cctctctgggcccatgccacctccaacatccactcgaccccttggaatttcggtggagaggagcagaggttgt
cctggcgtggtttaggtagtgtgagaggggtacccggggatcttgctaccagtggaacagccactaaggattc
tgcagtgagagcagagggccagctaagtggtactctcccagagactgtctgactcacgccaccccctccacct
tggacacaggacgctgtggtttctgagccaggtacaatgactcctttcggtaagtgcagtggaagctgtacac
tgcccaggcaaagcgtccgggcagcgtaggcgggcgactcagatcccagccagtggacttagcccctgtttgc
tcctccgataactggggtgaccttggttaatattcaccagcagcctcccccgttgcccctctggatccactgc
ttaaatacggacgaggacagggccctgtctcctcagcttcaggcaccaccactgacctgggacagtgaatgat
ccccctgatctgcggcc
- FVIIIco19SQ AAV construct sequence
>SEQ ID NO: 34
TTGGCCACTCCCTCTCTGCGCGCTCGCTCGCTCACTGAGGCCGGGCGACCAAAGGTCGCCCGACGCCCGGGCT
TTGCCCGGGCGGCCTCAGTGAGCGAGCGAGCGCGCAGAGAGGGAGTGGCCAACTCCATCACTAGGGGTTCC
TTTAATtaacccagccagtggacttagcccctgtttgctcctccgataactggggtgaccttggttaatattc
accagcagcctccccctcagcttcaggcaccaccactgacctgggacagtgaatcgcgccaccatgaagctgc
tcgcagcaactgtgctactcctcaccatctgcagccttgaaggagctactagaagatattatcttggggcagt
ggagctgagctgggactacatgcagtctgacctgggagaactgcctgtggatgccagatttccccctcgagtg
cccaagagcttcccctttaacacctcagtggtgtacaagaagaccctgtttgtggagtttacagaccatctct
tcaacattgctaagcccagacctccctggatgggcctgctgggccctaccatccaagctgaagtgtatgacac
tgttgtgatcacactcaagaacatggcctcccatcctgtgtccctgcatgcagtgggagtctcctactggaag
gcctcagaaggagcagagtatgatgaccagaccagccagagagagaaggaggatgacaaggtgtttcctggag
ggagccacacctatgtgtggcaggtgctgaaggagaatggacctatggccagtgaccctctgtgtcttaccta
ttcctacctgtcacatgtggatctggtgaaggacctgaacagtggcctgattggggctctgctggtttgcaga
gaaggcagcttggccaaggagaagacccaaaccctgcacaagttcatcctgctgtttgctgtgtttgatgagg
ggaaatcatggcactcagagaccaagaacagcctcatgcaggatagggatgctgccagtgccagggcttggcc
caagatgcacactgtgaatggctatgtgaatagaagcctgcctgggctgataggctgtcacagaaaatctgtg
tactggcatgtgattggcatgggcaccacacctgaggtgcactccattttcctggagggccacaccttccttg
tgagaaaccacagacaagcttccctggagatcagcccaatcacctttctgactgctcaaaccctcctgatgga
tctgggccagttcctgctgttctgtcatatctcctcacaccagcatgatggaatggaagcttatgtcaaggtg
gactcctgcccagaggaaccacagctcagaatgaagaacaatgaggaggctgaggactatgatgatgacctga
cagactctgaaatggatgtggtcagatttgatgatgacaacagcccttcattcatccaaatcagatctgtggc
caagaagcatcccaagacctgggtgcactacatagctgctgaggaggaggactgggactatgcccctctggtc
ctggcccctgatgacagaagctataaaagccagtacctgaataatggcccccagagaattggcagaaagtaca
agaaagtcagattcatggcttacactgatgagaccttcaaaaccagggaagccatccagcatgagtcaggcat
cctgggccccctgctgtatggggaggttggagataccctgctgattatcttcaaaaaccaggcaagcaggccc
tacaatatctaccctcatggcatcactgatgtcaggccactgtattccagaagactgcctaagggggtgaagc
acctgaaggacttcccaatcctgccaggggagattttcaaatacaagtggacagtgactgtggaggatggacc
aaccaagtcagatcctagatgtctgaccagatactactccagctttgtgaacatggagagagacctggcctct
ggcctgattggccctctgctgatctgctataaagagtcagtggaccagagaggcaaccagatcatgagtgaca
aaagaaatgtgatcttgttctcagtgtttgatgagaatagatcttggtacctcacagaaaacatccagaggtt
cctgcccaatccagctggggtgcagctggaagatccagaattccaggccagcaacatcatgcatagcatcaat
ggttatgtctttgacagcctgcagctgtcagtgtgtctgcatgaagttgcttactggtatattctgtccattg
gagcccagacagacttcctgtctgtcttcttctctggctacacctttaaacacaagatggtgtatgaggacac
cctgaccctgttccctttctctggggaaacagtgttcatgtccatggaaaaccctggactgtggatcctgggc
tgccataacagtgacttcagaaacagaggcatgacagccctgctcaaggtgtccagctgtgataagaacacag
gagactactatgaggatagctatgaggacatcagtgcttacctgctgagcaagaataatgccattgaacccag
gtcattttcccaaaatccccctgtgctgaaaaggcaccagagggagatcacgcgtaccaccctgcagagtgac
caggaggaaattgattatgatgacaccatctctgtggaaatgaaaaaggaggattttgacatctatgatgagg
atgagaaccagagccctagaagcttccagaaaaagactagacactacttcattgctgcagtggagagactctg
ggattatggcatgagctccagcccccatgtgctgagaaatagagctcagagtggcagtgtgccacagttcaag
aaggtggtgtttcaggagttcactgatggctccttcacacaaccactttacagaggagaactgaatgagcacc
tgggcctcctgggcccctacatcagggctgaagtggaggataacattatggtcacatttaggaatcaggcttc
cagaccctactccttttattcctcactcatttcctatgaggaggaccagaggcagggagctgagcccagaaaa
aattttgtgaaacccaatgaaaccaagacctacttctggaaggtgcagcaccatatggcccctaccaaggatg
aatttgactgcaaggcttgggcttacttttctgatgtggaccttgagaaagatgtgcattcaggcctcattgg
gccactgctggtgtgccacaccaatacactgaaccctgctcatgggagacaggtcacagtgcaggagtttgca
ctcttctttaccatctttgatgagaccaagtcctggtatttcactgagaacatggagaggaactgcagggccc
cttgtaacatccagatggaggatcccaccttcaaggaaaactacagattccatgccatcaatggctacatcat
ggacaccctgccaggcctggtgatggcccaggaccagaggatcaggtggtacctcctgtctatgggcagcaat
gaaaatatccacagcattcacttctctggacatgtgtttactgtgaggaagaaggaggaatacaagatggctc
tgtacaacctctaccctggggtgtttgaaacagtggagatgctgccctccaaggctggcatctggagagtgga
atgtctgattggggagcatctgcatgctggcatgagcacactgttcctggtgtattccaacaagtgccagacc
ccactgggcatggcctcaggacatatcagggacttccagatcactgctagtggacaatatggacagtgggcac
ccaagctggccagactgcactactcaggctccatcaatgcctggagtaccaaggagcccttcagctggatcaa
ggtggacctgctggcccccatgattatacatggcatcaagacccagggagctagacagaagttcagctccctg
tacatctcccaattcatcatcatgtactctctggatggcaagaaatggcagacctacagaggcaatagcactg
gcaccctgatggtgttttttggaaatgttgactcttctggcatcaagcacaacatcttcaacccccccatcat
tgccagatatatcaggctccaccccacccactactccataaggagcaccctgagaatggagctgatgggctgt
gacctgaattcctgctccatgcccctgggcatggaatccaaggcaatctctgatgcacagatcacagcctcct
cctacttcaccaacatgtttgcaacctggagcccctccaaggccagactgcacctgcaGGGCAGGTCCAATGC
CTTGGAGACACAAGTGAACAACCCAAAGGAGTGGCTGCAGGTGGACTTCCAGAAGACCATGAAAGTGACTGGA
GTGACCACCCAGGGAGTGAAATCCCTGCTCACTAGCATGTATGTGAAGGAATTCCTGATCAGTAGCTCTCAAG
ATGGCCACCAGTGGACCCTGTTCTTCCAGAATGGCAAGGTGAAGGTGTTTCAGGGCAACCAGGATTCCTTCAC
CCCTGTGGTGAATAGCCTGGATCCCCCACTGCTGACCAGATACCTGAGAATCCACCCCCAGTCCTGGGTTCAC
CAGATTGCCCTGAGAATGGAGGTGCTGGGCTGTGAGGCCCAGGACCTGTACTGAAATAAAAGATCTTTATTTT
CATTAGATCTGTGTGTTGGTTTTTTGTGTGAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGCCCGGGCGGCCTCAGTGAGC
GAGCGAGCGCGCAGAGAGGGAGTGGCCAA
FRE75 TRE
>SEQ ID NO: 35
gcaaacattgcaagcagcaaacagtggacttagcccctgtttgctcctccgataactggggtgaccttggtta
atattcaccagcagcctccccccccagccagtggacttagcccctgtttgctcctccgataactggggtgacc
ttggttaatattcaccagcagcctccccctcagcttcaggcaccaccactgacctgggacagtgaatc
FRE46 TRE
>SEQ ID NO: 36
agtggacttagcccctgtttgctcctccgataactggggtgaccttggttaatattcaccagcagcctccccc
FRE47 TRE
>SEQ ID NO: 37
agtggacttagcccctgtttgctcctccgataactggggtgaccttggttaatattcaccagcagcctccccc
tcagct

Numbered Aspects of the Invention
1. A transcription regulatory element comprising a core nucleotide sequence which comprises or consists of a nucleotide sequence having at least 95% identity to SEQ ID NO: 2, or a nucleotide sequence which differs from SEQ ID NO: 2 by a single nucleotide, and wherein the transcription regulatory element is between 80 and 280 nucleotides in length; optionally wherein the transcription regulatory element is between 80 and 225 nucleotides in length.
2. The transcription regulatory element of aspect 1, further comprising a nucleotide sequence located 3′ to the core nucleotide sequence.
3. The transcription regulatory element of aspect 2, wherein the nucleotide sequence located 3′ to the core nucleotide sequence comprises one or more transcription start sites (TSS).
4. The transcription regulatory element of aspect 3, wherein the one or more TSS comprise or consist of a nucleotide sequence according to:
   a. SEQ ID NO: 6, or a nucleotide sequence which differs from SEQ ID NO: 6 by a single nucleotide;
   b. SEQ ID NO: 7, or a nucleotide sequence which differs from SEQ ID NO: 7 by a single nucleotide; and/or
   c. SEQ ID NO: 8, or a nucleotide sequence which differs from SEQ ID NO: 8 by a single nucleotide.
5. The transcription regulatory element of any one of aspects 2 to 4, wherein the nucleotide sequence located 3′ to the core nucleotide sequence comprises:
   a. a nucleotide sequence according to SEQ ID NO: 6, or a nucleotide sequence which differs from SEQ ID NO: 6 by a single nucleotide; or
   b. a nucleotide sequence having at least 90% identity to SEQ ID NO: 9, or a nucleotide sequence which differs from SEQ ID NO: 9 by a single nucleotide; or
   c. a nucleotide sequence having at least 90% identity to SEQ ID NO: 10, or a nucleotide sequence which differs from SEQ ID NO: 10 by a single nucleotide.
6. The transcription regulatory element of any one of aspects 2 to 5, wherein the nucleotide sequence located 3′ to the core nucleotide sequence further comprises a nucleotide sequence defined by SEQ ID NO: 11, or a nucleotide sequence which differs from SEQ ID NO: 11 by a single nucleotide.
7. The transcription regulatory element of any one of aspects 2 to 6, wherein the nucleotide sequence located 3′ to the core nucleotide sequence is shorter than 50 nucleotides; optionally is shorter than 40 nucleotides; and optionally is shorter than 30 nucleotides.
8. The transcription regulatory element of any one of aspects 2 to 7, wherein the nucleotide sequence located 3′ to the core nucleotide sequence comprises or consists of a nucleotide sequence selected from the group consisting of:
   a. a nucleotide sequence having at least 90% or at least 95% identity to SEQ ID NO: 10, or a nucleotide sequence which differs from SEQ ID NO: 10 by a single nucleotide;
   b. a nucleotide sequence having at least 90% or at least 95% identity to SEQ ID NO: 12, or a nucleotide sequence which differs from SEQ ID NO: 12 by a single nucleotide; and
   c. a nucleotide sequence having at least 90% or at least 95% identity to SEQ ID NO: 13, or a nucleotide sequence which differs from SEQ ID NO: 13 by a single nucleotide.
9. The transcription regulatory element of any one of the preceding aspects, further comprising a nucleotide sequence located 5′ to the core nucleotide sequence.
10. The transcription regulatory element of aspect 9, wherein the nucleotide sequence located 5′ to the core nucleotide sequence comprises:
   a. a nucleotide sequence comprising at least 10, at least 15, or at least 20 consecutive nucleotides of SEQ ID NO: 14;
   b. a nucleotide sequence having at least 90% or at least 95% identity to SEQ ID NO: 14, or a nucleotide sequence which differs from SEQ ID NO: 14 by a single nucleotide;
   c. a nucleotide sequence comprising at least 10, at least 15, or at least 20 consecutive nucleotides of SEQ ID NO: 15;
   d. a nucleotide sequence having at least 90% or at least 95% identity to SEQ ID NO: 15, or a nucleotide sequence which differs from SEQ ID NO: 15 by a single nucleotide;
   e. a nucleotide sequence comprising at least 10, at least 15, at least 20, at least 30, at least 40, at least 50, at least 60, at least 70, at least 80 or at least 90 consecutive nucleotides of SEQ ID NO: 16;
   f. a nucleotide sequence having at least 90% or at least 95% identity to SEQ ID NO: 16, or a nucleotide sequence which differs from SEQ ID NO: 16 by a single nucleotide;
      and/or
   g. a nucleotide sequence defined by SEQ ID NO: 17, or a nucleotide sequence which differs from SEQ ID NO: 17 by a single nucleotide.
11. The transcription regulatory element of aspect 9 or aspect 10, wherein the nucleotide sequence located 5′ to the core nucleotide sequence has less than 60% identity to a nucleotide sequence comprising at least 20, at least 25, at least 30, at least 35, at least 40 or 45 consecutive nucleotides of SEQ ID NO: 4.
12. The transcription regulatory element of aspect 11, wherein the nucleotide sequence located 5′ to the core nucleotide sequence has less than 50% identity to a nucleotide sequence comprising at least 20, at least 25, at least 30, at least 35, at least 40 or 45 consecutive nucleotides of SEQ ID NO: 4; optionally wherein it has less than 45% identity; optionally wherein it has less than 40% identity; and optionally wherein it has less than 30% identity.
13. The transcription regulatory element of any one of aspects 9 to 12, wherein the nucleotide sequence located 5′ to the core nucleotide sequence is shorter than 110 nucleotides; optionally is shorter than 100 nucleotides; optionally is shorter than 50 nucleotides; and optionally is shorter than 10 nucleotides.
14. The transcription regulatory element of any one of aspects 9 to 13, wherein the nucleotide sequence located 5′ to the core nucleotide sequence is between 5 and 110 nucleotides in length.
15. The transcription regulatory element of any one of aspects 9 to 14, wherein the nucleotide sequence located 5′ to the core nucleotide sequence is at least 7 nucleotides in length.
16. The transcription regulatory element of any one of aspects 9 to 15, wherein the nucleotide sequence located 5′ to the core nucleotide sequence is 102 nucleotides or less in length.
17. The transcription regulatory element of any one of aspects 9 to 16, wherein the nucleotide sequence located 5′ to the core nucleotide sequence comprises a nucleotide sequence selected from the group consisting of:
   a. a nucleotide sequence having at least 90% or at least 95% identity to SEQ ID NO: 18, or a nucleotide sequence which differs from SEQ ID NO: 18 by a single nucleotide;
   b. a nucleotide sequence having at least 90% or at least 95% identity to SEQ ID NO: 19, or a nucleotide sequence which differs from SEQ ID NO: 19 by a single nucleotide;
   c. a nucleotide sequence having at least 90% or at least 95% identity to SEQ ID NO: 20, or a nucleotide sequence which differs from 90% or SEQ ID NO: 20 by a single nucleotide
   d. a nucleotide sequence having at least 90% or at least 95% identity to SEQ ID NO: 21, or a nucleotide sequence which differs from SEQ ID NO: 21 by a single nucleotide;
   e. a nucleotide sequence having at least 90% or at least 95% identity to SEQ ID NO: 22, or a nucleotide sequence which differs from SEQ ID NO: 22 by a single nucleotide; and
   f. a nucleotide sequence according to SEQ ID NO: 23, or which differs from SEQ ID NO: 23 by a single nucleotide.
18. The transcription regulatory element of any one of the preceding aspects, wherein the transcription regulatory element:
   a. does not comprise a nucleotide sequence according to SEQ ID NO: 4, or does not comprise at least 20, at least 30 or at least 40 consecutive nucleotides of SEQ ID NO: 4;
      and/or
   b. does not comprise a nucleotide sequence according to SEQ ID NO: 5, or does not comprise at least 20, at least 30 or at least 40 consecutive nucleotides of SEQ ID NO: 5.
19. The transcription regulatory element of aspect 18, which:
   a. does not comprise a nucleotide sequence having at least 90% or at least 95% identity to SEQ ID NO: 4;
      and/or
   b. does not comprise a nucleotide sequence having at least 90% or at least 95% identity to SEQ ID NO: 5.
20. The transcription regulatory element of any one of the preceding aspects, which is shorter than 200 nucleotides; optionally which is shorter than 150 nucleotides; and optionally which is shorter than 125 nucleotides.
21. The transcription regulatory element of any one of the preceding aspects, which is at least 85 nucleotides in length, optionally which is at least 100 nucleotides in length, optionally which is at least 110 nucleotides in length.
22. A transcription regulatory element comprising a core nucleotide sequence which comprises or consists of a nucleotide sequence having at least 95% identity to SEQ ID NO: 2, or a nucleotide sequence which differs from SEQ ID NO: 2 by a single nucleotide, wherein the transcription regulatory element:
   a. does not comprise at least 20, at least 30 or at least 40 consecutive nucleotides of SEQ ID NO: 4;
      and/or
   b. does not comprise at least 20, at least 30 or at least 40 consecutive nucleotides of SEQ ID NO: 5;
   and wherein the transcription regulatory element is between 80 and 280 nucleotides in length.
23. The transcription regulatory element of aspect 22, which:
   a. does not comprise a nucleotide sequence having at least 90%, or at least 95% or 100% identity to SEQ ID NO: 4;
      and/or
   b. does not comprise a nucleotide sequence having at least 90%, or at least 95% or 100% identity to SEQ ID NO: 5.
24. The transcription regulatory element of aspect 22 or aspect 23, further comprising a nucleotide sequence located 3′ to the core nucleotide sequence.
25. The transcription regulatory element of aspect 24, wherein the nucleotide sequence located 3′ to the core nucleotide sequence comprises one or more transcription stop sites (TSS).
26. The transcription regulatory element of aspect 25, wherein the one or more TSS comprise a nucleotide sequence according to:
   a. SEQ ID NO: 6, or a nucleotide sequence which differs from SEQ ID NO: 6 by a single nucleotide;
   b. SEQ ID NO: 7, or a nucleotide sequence which differs from SEQ ID NO: 7 by a single nucleotide; and/or
   c. SEQ ID NO: 8, or a nucleotide sequence which differs from SEQ ID NO: 8 by a single nucleotide.
27. The transcription regulatory element of aspect 24, aspect 25 or aspect 26, wherein the nucleotide sequence located 3′ to the core nucleotide sequence comprises:
   a. a nucleotide sequence according to SEQ ID NO: 6, or a nucleotide sequence which differs from SEQ ID NO: 6 by a single nucleotide; or
   b. a nucleotide sequence having at least 90% identity to SEQ ID NO: 9, or a nucleotide sequence which differs from SEQ ID NO: 9 by a single nucleotide; or
   c. a nucleotide sequence having at least 90% identity to SEQ ID NO: 10, or a nucleotide sequence which differs from SEQ ID NO: 10 by a single nucleotide.
28. The transcription regulatory element of any one of aspects 24 to 27, wherein the nucleotide sequence located 3′ to the core nucleotide sequence further comprises a nucleotide sequence defined by SEQ ID NO: 11, or a nucleotide sequence which differs from SEQ ID NO: 11 by a single nucleotide.
29. The transcription regulatory element of any one of aspects 24 to 28, wherein the nucleotide sequence located 3′ to the core nucleotide sequence is shorter than 50 nucleotides; optionally is shorter than 40 nucleotides; and optionally is shorter than 30 nucleotides.
30. The transcription regulatory element of any one of aspects 24 to 29, wherein the nucleotide sequence located 3′ to the core nucleotide sequence comprises or consists of a nucleotide sequence selected from the group consisting of:
   a. a nucleotide sequence having at least 90% or at least 95% identity to SEQ ID NO: 10, or a nucleotide sequence which differs from SEQ ID NO: 10 by a single nucleotide;
   b. a nucleotide sequence having at least 90% or at least 95% identity to SEQ ID NO:12, or a nucleotide sequence which differs from SEQ ID NO: 12 by a single nucleotide; and
   c. a nucleotide sequence having at least 90% or at least 95% identity to SEQ ID NO: 13, or a nucleotide sequence which differs from SEQ ID NO: 13 by a single nucleotide.
31. The transcription regulatory element of any one of aspects 22 to 30, further comprising a nucleotide sequence located 5′ to the core nucleotide sequence.
32. The transcription regulatory element of aspect 31, wherein the nucleotide sequence located 5′ to the core nucleotide sequence comprises:
   a. a nucleotide sequence comprising at least 10, at least 15, or at least 20 consecutive nucleotides of SEQ ID NO: 14;
   b. a nucleotide sequence having at least 90% or at least 95% identity to SEQ ID NO: 14, or a nucleotide sequence which differs from SEQ ID NO: 14 by a single nucleotide;
   c. a nucleotide sequence comprising at least 10, at least 15, or at least 20 consecutive nucleotides of SEQ ID NO: 15;
   d. a nucleotide sequence having at least 90% or at least 95% identity to SEQ ID NO: 15, or a nucleotide sequence which differs from SEQ ID NO: 15 by a single nucleotide;
   e. a nucleotide sequence comprising at least 10, at least 15, at least 20, at least 30, at least 40, at least 50, at least 60, at least 70, at least 80 or at least 90 consecutive nucleotides of SEQ ID NO: 16;
   f. a nucleotide sequence having at least 90% or at least 95% identity to SEQ ID NO: 16, or a nucleotide sequence which differs from SEQ ID NO: 16 by a single nucleotide;
      and/or
   g. a nucleotide sequence defined by SEQ ID NO: 17, or a nucleotide sequence which differs from SEQ ID NO: 17 by a single nucleotide.
33. The transcription regulatory element of aspect 31 or 32, wherein the nucleotide sequence located 5′ to the core nucleotide sequence has less than 60% identity to a nucleotide sequence comprising at least 20, at least 25, at least 30, at least 35, at least 40 or 45 consecutive nucleotides of SEQ ID NO: 4.
34. The transcription regulatory element of aspect 33, wherein the nucleotide sequence located 5′ to the core nucleotide sequence has less than 50% identity to a nucleotide sequence comprising at least 20, at least 25, at least 30, at least 35, at least 40 or 45 consecutive nucleotides of SEQ ID NO: 4; optionally wherein it has less than 45% identity; optionally wherein it has less than 40% identity; and optionally wherein it has less than 30% identity.
35. The transcription regulatory element of any one of aspects 31 to 34, wherein the nucleotide sequence located 5′ to the core nucleotide sequence is shorter than 110 nucleotides; optionally is shorter than 100 nucleotides; optionally is shorter than 50 nucleotides; and optionally is shorter than 10 nucleotides.
36. The transcription regulatory element of any one of aspects 31 to 35, wherein the nucleotide sequence located 5′ to the core nucleotide sequence is between 5 and 110 nucleotides in length.
37. The transcription regulatory element of any one of aspects 31 to 36, wherein the nucleotide sequence located 5′ to the core nucleotide sequence is at least 7 nucleotides in length.
38. The transcription regulatory element of any one of aspects 31 to 37, wherein the nucleotide sequence located 5′ to the core nucleotide sequence is 102 nucleotides or less in length.
39. The transcription regulatory element of any one of aspects 31 to 38, wherein the nucleotide sequence located 5′ to the core nucleotide sequence comprises a nucleotide sequence selected from the group consisting of:
   a. a nucleotide sequence having at least 90% or at least 95% identity to SEQ ID NO: 18, or a nucleotide sequence which differs from SEQ ID NO: 18 by a single nucleotide;
   b. a nucleotide sequence having at least 90% or at least 95% identity to SEQ ID NO: 19, or a nucleotide sequence which differs from SEQ ID NO: 19 by a single nucleotide;
   c. a nucleotide sequence having at least 90% or at least 95% identity to SEQ ID NO: 20, or a nucleotide sequence which differs from SEQ ID NO: 20 by a single nucleotide;
   d. a nucleotide sequence having at least 90% or at least 95% identity to SEQ ID NO: 21, or a nucleotide sequence which differs from SEQ ID NO: 21 by a single nucleotide;
   e. a nucleotide sequence having at least 90% or at least 95% identity to SEQ ID NO: 22, or a nucleotide sequence which differs from SEQ ID NO: 22 by a single nucleotide; and
   f. a nucleotide sequence according to SEQ ID NO: 23, or which differs from SEQ ID NO: 23 by a single nucleotide.
40. The transcription regulatory element of any one of aspects 22 to 39, which is shorter than 200 nucleotides; optionally which is shorter than 150 nucleotides; and optionally which is shorter than 125 nucleotides.
41. The transcription regulatory element of any one of aspects 22 to 40, which is at least 85 nucleotides in length, optionally which is at least 100 nucleotides in length, and optionally which is at least 110 nucleotides in length.
42. A transcription regulatory element comprising:
   a. a core nucleotide sequence which comprises or consists of a nucleotide sequence having at least 95% identity to SEQ ID NO: 2, or a nucleotide sequence which differs from SEQ ID NO: 2 by a single nucleotide; and
   b. a nucleotide sequence which is located 5′ to the core nucleotide sequence and which has less than 60% identity to a nucleotide sequence comprising at least 20, at least 25, at least 30, at least 35, at least 40 or 45 consecutive nucleotides of SEQ ID NO: 4;
   wherein the transcription regulatory element is between 80 and 280 nucleotides in length.
43. The transcription regulatory element of aspect 42, wherein the nucleotide sequence located 5′ to the core nucleotide sequence comprises:
   a. a nucleotide sequence comprising at least 10, at least 15, or at least 20 consecutive nucleotides of SEQ ID NO: 14;
   b. a nucleotide sequence having at least 90% or at least 95% identity to SEQ ID NO: 14, or a nucleotide sequence which differs from SEQ ID NO: 14 by a single nucleotide;
   c. a nucleotide sequence comprising at least 10, at least 15, or at least 20 consecutive nucleotides of SEQ ID NO: 15;
   d. a nucleotide sequence having at least 90% or at least 95% identity to SEQ ID NO: 15, or a nucleotide sequence which differs from SEQ ID NO: 15 by a single nucleotide;
   e. a nucleotide sequence comprising at least 10, at least 15, at least 20, at least 30, at least 40, at least 50, at least 60 at least 70, at least 80, or at least 90 consecutive nucleotides of SEQ ID NO: 16;
   f. a nucleotide sequence having at least 90% or at least 95% identity to SEQ ID NO: 16, or a nucleotide sequence which differs from SEQ ID NO: 16 by a single nucleotide;
      and/or
   g. a nucleotide sequence defined by SEQ ID NO: 17, or a nucleotide sequence which differs from SEQ ID NO: 17 by a single nucleotide.
44. The transcription regulatory element of aspect 42 or aspect 43, wherein the nucleotide sequence located 5′ to the core nucleotide sequence has less than 50% identity to a nucleotide sequence comprising at least 20, at least 25, at least 30, at least 35, at least 40 or 45 consecutive nucleotides of SEQ ID NO: 4; optionally wherein it has less than 45% identity; optionally wherein it has less than 40% identity; and optionally wherein it has less than 30% identity.
45. The transcription regulatory element of any one of aspects 42 to 44, wherein the nucleotide sequence located 5′ to the core nucleotide sequence is shorter than 110 nucleotides; optionally is shorter than 100 nucleotides; optionally is shorter than 50 nucleotides; and optionally is shorter than 10 nucleotides.
46. The transcription regulatory element of any one of aspects 42 to 45, wherein the nucleotide sequence located 5′ to the core nucleotide sequence is between 5 and 110 nucleotides in length.
47. The transcription regulatory element of any one of aspects 42 to 46, wherein the nucleotide sequence located 5′ to the core nucleotide sequence is at least 7 nucleotides in length.
48. The transcription regulatory element of any one of aspects 42 to 47, wherein the nucleotide sequence located 5′ to the core nucleotide sequence is 102 nucleotides or less in length.
49. The transcription regulatory element of any one of aspects 42 to 48, wherein the nucleotide sequence located 5′ to the core nucleotide sequence comprises a nucleotide sequence selected from the group consisting of:
   a. a nucleotide sequence having at least 90% or at least 95% identity to SEQ ID NO: 18, or a nucleotide sequence which differs from SEQ ID NO: 18 by a single nucleotide;
   b. a nucleotide sequence having at least 90% or at least 95% identity to SEQ ID NO: 19, or a nucleotide sequence which differs from SEQ ID NO: 19 by a single nucleotide;
   c. a nucleotide sequence having at least 90% or at least 95% identity to SEQ ID NO: 20, or a nucleotide sequence which differs from SEQ ID NO: 20 by a single nucleotide;
   d. a nucleotide sequence having at least 90% or at least 95% identity to SEQ ID NO: 21, or a nucleotide sequence which differs from SEQ ID NO: 21 by a single nucleotide;
   e. a nucleotide sequence having at least 90% or at least 95% identity to SEQ ID NO: 22, or a nucleotide sequence which differs from SEQ ID NO: 22 by a single nucleotide; and
   f. a nucleotide sequence according to SEQ ID NO: 23, or which differs from SEQ ID NO: 23 by a single nucleotide.
50. The transcription regulatory element of any one of aspects 42 to 49, further comprising a nucleotide sequence which is located 3′ to the core nucleotide sequence.
51. The transcription regulatory element of aspect 50, wherein the nucleotide sequence which is located 3′ to the core nucleotide sequence comprises a nucleotide sequence according to SEQ ID NO: 6, or a nucleotide sequence which differs from SEQ ID NO: 6 by a single nucleotide.
52. The transcription regulatory element of aspect 51, wherein the nucleotide sequence located 3′ to the core nucleotide sequence comprises:
   a. a nucleotide sequence according to SEQ ID NO: 6, or a nucleotide sequence which differs from SEQ ID NO: 6 by a single nucleotide; or
   b. a nucleotide sequence having at least 90% identity to SEQ ID NO: 9, or a nucleotide sequence which differs from SEQ ID NO: 9 by a single nucleotide; or
   c. a nucleotide sequence having at least 90% identity to SEQ ID NO: 10, or a nucleotide sequence which differs from SEQ ID NO: 10 by a single nucleotide.
53. The transcription regulatory element of any one of aspects 50 to 52, wherein the nucleotide sequence located 3′ to the core nucleotide sequence further comprises a nucleotide sequence defined by SEQ ID NO: 11, or a nucleotide sequence which differs from SEQ ID NO: 11 by a single nucleotide.
54. The transcription regulatory element of any one of aspects 50 to 53, wherein the nucleotide sequence located 3′ to the core nucleotide sequence is shorter than 50 nucleotides; optionally is shorter than 40 nucleotides; and optionally is shorter than 30 nucleotides.
55. The transcription regulatory element of any one of aspects 50 to 54, wherein the nucleotide sequence located 3′ to the core nucleotide sequence comprises one or more transcription start sites (TSS).
56. The transcription regulatory element of aspect 55, wherein the one or more TSS comprise a nucleotide sequence according to:
   a. SEQ ID NO: 6, or a nucleotide sequence which differs from SEQ ID NO: 6 by a single nucleotide;
   b. SEQ ID NO: 7, or a nucleotide sequence which differs from SEQ ID NO: 7 by a single nucleotide; and/or
   c. SEQ ID NO: 8, or a nucleotide sequence which differs from SEQ ID NO: 8 by a single nucleotide.
57. The transcription regulatory element of any one of aspects 50 to 56, wherein the nucleotide sequence located 3′ to the core nucleotide sequence comprises or consists of a nucleotide sequence selected from the group consisting of:
   a. a nucleotide sequence having at least 90% or at least 95% identity to SEQ ID NO: 10, or a nucleotide sequence which differs from SEQ ID NO: 10 by a single nucleotide;
   b. a nucleotide sequence having at least 90% or at least 95% identity to SEQ ID NO:12, or a nucleotide sequence which differs from SEQ ID NO: 12 by a single nucleotide; and
   c. a nucleotide sequence having at least 90% or at least 95% identity to SEQ ID NO: 13, or a nucleotide sequence which differs from SEQ ID NO: 13 by a single nucleotide.
58. The transcription regulatory element of any one of aspects 42 to 57, wherein the transcription regulatory element:
   a. does not comprise a nucleotide sequence according to SEQ ID NO: 4, or does not comprise at least 20, at least 30 or at least 40 consecutive nucleotides of SEQ ID NO: 4;
      and/or
   b. does not comprise a nucleotide sequence according to SEQ ID NO: 5, or does not comprise at least 20, at least 30 or at least 40 consecutive nucleotides of SEQ ID NO: 5.
59. The transcription regulatory element of aspect 58, which:
   a. does not comprise a nucleotide sequence having at least 90%, or at least 95% identity to SEQ ID NO: 4;
      and/or
   b. does not comprise a nucleotide sequence having at least 90%, or at least 95% identity to SEQ ID NO: 5.
60. The transcription regulatory element of any one of aspects 42 to 59, which is shorter than 200 nucleotides; optionally which is shorter than 150 nucleotides; and optionally which is shorter than 125 nucleotides.
61. The transcription regulatory element of any one of aspects 42 to 60, which is at least 85 nucleotides in length, optionally which is at least 100 nucleotides in length, and optionally which is at least 110 nucleotides in length.
62. The transcription regulatory element of any one of the preceding aspects, wherein the transcription regulatory element terminates in a ten-nucleotide sequence selected from:
   a. acagtgaatc; or
   b. ctcctcagct.
63. The transcription regulatory element of any one the preceding aspects, wherein the core nucleotide sequence is 73-80 nucleotides in length.
64. The transcription regulatory element of any one of the preceding aspects, wherein the core nucleotide sequence comprises or consists of a nucleotide sequence having at least 95% identity, and optionally at least 98% identity, to SEQ ID NO: 2.
65. The transcription regulatory element of any one of the preceding aspects, wherein the core nucleotide sequence is identical to SEQ ID NO: 2.
66. The transcription regulatory element of any one of aspects 1 to 65 wherein the core nucleotide sequence comprises or consists of a nucleotide sequence which has at least 95% identity, and optionally at least 98% identity, to SEQ ID NO: 3.
67. The transcription regulatory element of aspect 66, wherein the core nucleotide sequence has at least 95% identity, and optionally at least 98% identity, to SEQ ID NO: 3.
68. The transcription regulatory element of aspect 67, wherein the core nucleotide sequence is identical to SEQ ID NO: 3.
69. The transcription regulatory element of any one of aspects 1 to 68, which has a nucleotide sequence that has at least 90% identity, optionally at least 95% identity or optionally at least 98% identity to a nucleotide sequence selected from the group consisting of:
   a. SEQ ID NO: 24;
   b. SEQ ID NO: 25;
   c. SEQ ID NO: 26;
   d. SEQ ID NO: 27;
   e. SEQ ID NO: 28; and
   f. SEQ ID NO: 29.
70. The transcription regulatory element of any one of aspects 1 to 63, which has a nucleotide sequence selected from the group consisting of:
   a. SEQ ID NO: 24;
   b. SEQ ID NO: 25;
   c. SEQ ID NO: 26;
   d. SEQ ID NO: 27;
   e. SEQ ID NO: 28; and
   f. SEQ ID NO: 29.
71. The transcription regulatory element of any one of the preceding aspects, wherein the transcription regulatory element comprises a promoter; optionally wherein the transcription regulatory element further comprises an enhancer.
72. The transcription regulatory element of aspect 71, wherein the promoter is liver-specific.
73. A polynucleotide comprising a transcription regulatory element of any one of the preceding aspects, wherein the transcription regulatory element is operably linked to a transgene optionally wherein the transgene encodes a human protein.
74. The transcription regulatory element of any one of aspects 1 to 72, wherein the transcription regulatory element is part of a vector comprising a transgene, optionally wherein the vector is a viral particle such as an AAV vector.
75. The polynucleotide of aspect 73 or the transcription regulatory element of aspect 74, wherein the transcription regulatory element expresses the transgene at 50% or better compared to a transcription regulatory element defined by SEQ ID NO: 1 or SEQ ID NO: 33.
76. The polynucleotide of aspect 73 or the transcription regulatory element of aspect 74, wherein the transcription regulatory element expresses the transgene at 80% or better compared to a transcription regulatory element defined by SEQ ID NO: 1 or SEQ ID NO: 33.
77. The polynucleotide of aspect 73 or the transcription regulatory element of aspect 74, wherein the transcription regulatory element expresses the transgene at 100% or better compared to a transcription regulatory element defined by SEQ ID NO: 1 or SEQ ID NO: 33; optionally wherein the transcription regulatory element expresses the transgene at 110% or better, 120% or better, 140% or better, or 150% or better compared to a transcription regulatory element defined by SEQ ID NO: 1 or SEQ ID NO: 33.
78. The polynucleotide or the transcription regulatory element of any one of aspects 75 to 77, wherein expression of the transgene is determined in vitro in Huh7 cells.
79. The polynucleotide or transcription regulatory element of any one of aspects 73 to 78, wherein the transgene encodes a protein or a non-translated RNA which optionally is an siRNA, or an miRNA, or a snRNA or an antisense RNA.
80. The polynucleotide or transcription regulatory element of any one of aspects 73 to 79, wherein the transgene is longer than 4 k nucleotides; and optionally wherein the transgene is longer than 4.2 k nucleotides.
81. The polynucleotide or transcription regulatory element of aspect 80, wherein the transgene is shorter than 4.5 k nucleotides, optionally wherein the transgene is shorter than 4.4 k nucleotides.
82. The polynucleotide or transcription regulatory element of any one of aspects 73 to 81, wherein the transgene encodes FVIII; optionally wherein the transgene encodes a truncated or modified FVIII; optionally wherein the transgene encodes a B-domain deleted FVIII.
83. A vector comprising a nucleotide sequence which comprises: (i) the transcription regulatory element of any one of aspects 1 to 72; and (ii) a transgene.
84. The vector of aspect 83, wherein the vector nucleotide sequence further comprises a nucleotide sequence encoding a signal peptide.
85. The vector of aspect 84, wherein the nucleotide sequence encoding the signal peptide is 50 to 100 nucleotides in length.
86. The vector of aspect 85, wherein the nucleotide sequence encoding the signal peptide is shorter than 80 nucleotides.
87. The vector of any one of aspects 83 to 86, which is a viral particle such as an AAV vector.
88. The vector of any one of aspects 83 to 87, wherein the transcription regulatory element expresses the transgene at 50% or better compared to a transcription regulatory element defined by SEQ ID NO: 1 or SEQ ID NO: 33.
89. The vector of any one of aspects 83 to 87, wherein the transcription regulatory element expresses the transgene at 80% or better compared to a transcription regulatory element defined by SEQ ID NO: 1 or SEQ ID NO: 33.
90. The vector of any one of aspects 83 to 87, wherein the transcription regulatory element expresses the transgene at 100% or better compared to a transcription regulatory element defined by SEQ ID NO: 1 or SEQ ID NO: 33; and optionally wherein the transcription regulatory element expresses the transgene at 110% or better, 120% or better, 140% or better, or 150% or better compared to a transcription regulatory element defined by SEQ ID NO: 1 or SEQ ID NO: 33.
91. The vector of any one of aspects 89 to 90, wherein expression of the transgene is determined in vitro in Huh7 cells.
92. The vector of any one of aspects 83 to 91, wherein the transgene encodes a protein or a non-translated RNA which optionally is an siRNA, or an miRNA, or a snRNA or an antisense RNA.
93. The vector of any one of aspects 83 to 92, wherein the transgene is longer than 4 k nucleotides; and optionally wherein the transgene is longer than 4.2 nucleotides.
94. The vector of aspect 93, wherein the transgene is shorter than 4.5 k nucleotides, optionally wherein the transgene is shorter than 4.4 k nucleotides.
95. The vector of any one of aspects 83 to 94, wherein the transgene encodes FVIII; optionally wherein the transgene encodes a truncated or modified FVIII; optionally wherein the transgene encodes a B-domain deleted FVIII.
96. The vector of any one of aspects 83 to 95 wherein the vector genome is shorter than 4.9 k nucleotides, and optionally wherein the vector genome is no shorter than 4.5 k nucleotides.
97. The vector of aspect 96, wherein the vector genome is around 4.7 k nucleotides in length.
98. The vector of any one of aspects 83 to 97 for use in a method of treatment, optionally wherein the method of treatment is a method of gene therapy and/or a method of treating Haemophilia A.
99. A method of treatment comprising administering an effective amount of the vector of any one of aspects 83 to 97 to a patient, optionally wherein the method of treatment is a method of gene therapy and/or a method of treating Haemophilia A.
100. Use of the vector of any one of aspects 83 to 97 in a method of treatment, optionally wherein the method of treatment is a method of gene therapy and/or a method of treating Haemophilia A.

1. A transcription regulatory element comprising a core nucleotide sequence which comprises or consists of a nucleotide sequence having at least 95% identity to SEQ ID NO: 2, or a nucleotide sequence which differs from SEQ ID NO: 2 by a single nucleotide; and wherein:
a. the transcription regulatory element is between 80 and 280 nucleotides in length and:
i) does not comprise a nucleotide sequence according to SEQ ID NO: 5, or does not comprise at least 20, at least 30 or at least 40 consecutive nucleotides of SEQ ID NO: 5; and/or
ii) does not comprise a nucleotide sequence according to SEQ ID NO: 4, or does not comprise at least 20, at least 30 or at least 40 consecutive nucleotides of SEQ ID NO: 4;
or
b. the transcription regulatory element is between 80 and 280 nucleotides in length and further comprises a nucleotide sequence which is located 5′ to the core nucleotide sequence and which has less than 60% identity to a nucleotide sequence comprising at least 20, at least 25, at least 30, at least 35, at least 40 or 45 consecutive nucleotides of SEQ ID NO: 4.

2. The transcription regulatory element as claimed in claim 1, which comprises or consists of a nucleotide sequence which has at least 90% identity, optionally at least 95% identity or optionally at least 98% identity, to a nucleotide sequence according to SEQ ID NO: 29.

3. The transcription regulatory element as claimed in claim 1, further comprising a nucleotide sequence located 3′ to the core nucleotide sequence, and optionally wherein:
a. the nucleotide sequence located 3′ to the core nucleotide sequence comprises one or more transcription start sites (TSS); optionally wherein the one or more TSS comprise or consist of a nucleotide sequence according to:
i. SEQ ID NO: 6, or a nucleotide sequence which differs from SEQ ID NO: 6 by a single nucleotide;
ii. SEQ ID NO: 7, or a nucleotide sequence which differs from SEQ ID NO: 7 by a single nucleotide; and/or
iii. SEQ ID NO: 8, or a nucleotide sequence which differs from SEQ ID NO: 8 by a single nucleotide;
and/or
b. the nucleotide sequence located 3′ to the core nucleotide sequence comprises:
i. a nucleotide sequence according to SEQ ID NO: 6, or a nucleotide sequence which differs from SEQ ID NO: 6 by a single nucleotide; or
ii. a nucleotide sequence having at least 90% identity to SEQ ID NO: 9, or a nucleotide sequence which differs from SEQ ID NO: 9 by a single nucleotide; or
iii. a nucleotide sequence having at least 90% identity to SEQ ID NO: 10, or a nucleotide sequence which differs from SEQ ID NO: 10 by a single nucleotide;
and/or
c. the nucleotide sequence located 3′ to the core nucleotide sequence further comprises a nucleotide sequence defined by SEQ ID NO: 11, or a nucleotide sequence which differs from SEQ ID NO: 11 by a single nucleotide;
and/or
d. the nucleotide sequence located 3′ to the core nucleotide sequence is shorter than 50 nucleotides; optionally is shorter than 40 nucleotides; and optionally is shorter than 30 nucleotides;
and/or
e. the nucleotide sequence located 3′ to the core nucleotide sequence comprises or consists of a nucleotide sequence selected from the group consisting of:
i. a nucleotide sequence having at least 90% or at least 95% identity to SEQ ID NO: 10, or a nucleotide sequence which differs from SEQ ID NO: 10 by a single nucleotide;
ii. a nucleotide sequence having at least 90% or at least 95% identity to SEQ ID NO:12, or a nucleotide sequence which differs from SEQ ID NO: 12 by a single nucleotide; and
iii. a nucleotide sequence having at least 90% or at least 95% identity to SEQ ID NO:
13, or a nucleotide sequence which differs from SEQ ID NO: 13 by a single nucleotide.

4. The transcription regulatory element as claimed in claim 1 or claim 3, further comprising a nucleotide sequence located 5′ to the core nucleotide sequence, and optionally wherein:
a. the nucleotide sequence located 5′ to the core nucleotide sequence comprises:
i. a nucleotide sequence comprising at least 10, at least 15, or at least 20 consecutive nucleotides of SEQ ID NO: 14;
ii. a nucleotide sequence having at least 90% or at least 95% identity to SEQ ID NO: 14, or a nucleotide sequence which differs from SEQ ID NO: 14 by a single nucleotide;
iii. a nucleotide sequence comprising at least 10, at least 15, or at least 20 consecutive nucleotides of SEQ ID NO: 15;
iv. a nucleotide sequence having at least 90% or at least 95% identity to SEQ ID NO: 15, or a nucleotide sequence which differs from SEQ ID NO: 15 by a single nucleotide;
v. a nucleotide sequence comprising at least 10, at least 15, at least 20, at least 30, at least 40, at least 50, at least 60, at least 70, at least 80 or at least 90 consecutive nucleotides of SEQ ID NO: 16;
vi. a nucleotide sequence having at least 90% or at least 95% identity to SEQ ID NO:
16, or a nucleotide sequence which differs from SEQ ID NO: 16 by a single nucleotide; and/or
vii. a nucleotide sequence defined by SEQ ID NO: 17, or a nucleotide sequence which differs from SEQ ID NO: 17 by a single nucleotide;
and/or
b. the nucleotide sequence located 5′ to the core nucleotide sequence has less than 60% identity to a nucleotide sequence comprising at least 20, at least 25, at least 30, at least 35, at least 40 or 45 consecutive nucleotides of SEQ ID NO: 4; and optionally wherein the nucleotide sequence located 5′ to the core nucleotide sequence has less than 50% identity to a nucleotide sequence comprising at least 20, at least 25, at least 30, at least 35, at least 40 or 45 consecutive nucleotides of SEQ ID NO: 4; optionally wherein it has less than 45% identity; optionally wherein it has less than 40% identity; and optionally wherein it has less than 30% identity;
and/or
c. the nucleotide sequence located 5′ to the core nucleotide sequence is shorter than 110 nucleotides; optionally is shorter than 100 nucleotides; optionally is shorter than 50 nucleotides; and optionally is shorter than 10 nucleotides;
and/or
d. the nucleotide sequence located 5′ to the core nucleotide sequence is between 5 and 110 nucleotides in length;
and/or
e. the nucleotide sequence located 5′ to the core nucleotide sequence is at least 7 nucleotides in length;
and/or
f. the nucleotide sequence located 5′ to the core nucleotide sequence is 102 nucleotides or less in length;
and/or
g. the nucleotide sequence located 5′ to the core nucleotide sequence comprises a nucleotide sequence selected from the group consisting of:
i. a nucleotide sequence having at least 90% or at least 95% identity to SEQ ID NO: 18, or a nucleotide sequence which differs from SEQ ID NO: 18 by a single nucleotide;
ii. a nucleotide sequence having at least 90% or at least 95% identity to SEQ ID NO: 19, or a nucleotide sequence which differs from SEQ ID NO: 19 by a single nucleotide;
iii. a nucleotide sequence having at least 90% or at least 95% identity to SEQ ID NO: 20, or a nucleotide sequence which differs from SEQ ID NO: 20 by a single nucleotide;
iv. a nucleotide sequence having at least 90% or at least 95% identity to SEQ ID NO: 21, or a nucleotide sequence which differs from SEQ ID NO: 21 by a single nucleotide;
v. a nucleotide sequence having at least 90% or at least 95% identity to SEQ ID NO: 22, or a nucleotide sequence which differs from SEQ ID NO: 22 by a single nucleotide; and
vi. a nucleotide sequence according to SEQ ID NO: 23, or a nucleotide sequence which differs from SEQ ID NO: 23 by a single nucleotide.

5. The transcription regulatory element as claimed in claim 1, claim 3 or claim 4, which is shorter than 200 nucleotides; optionally which is shorter than 150 nucleotides; and optionally which is shorter than 125 nucleotides.

6. The transcription regulatory element as claimed in claim 1 or in any one of claims 3 to 5, which is at least 85 nucleotides in length, optionally which is at least 100 nucleotides in length, and optionally which is at least 110 nucleotides in length.

7. The transcription regulatory element as claimed in claim 1 or in any one of claims 3 to 6, wherein the transcription regulatory element terminates in a ten-nucleotide sequence selected from:
a. acagtgaatc; or
b. ctcctcagct.

8. The transcription regulatory element as claimed in claim 1 or in any one of claims 3 to 7, wherein:
a. the core nucleotide sequence is 73-80 nucleotides in length;
and/or
b. the core nucleotide sequence comprises or consists of a nucleotide sequence which has at least 95% identity, and optionally at least 98% identity, to SEQ ID NO: 2;
and/or
c. the core nucleotide sequence is identical to SEQ ID NO: 2;
and/or
d. the core nucleotide sequence comprises or consists of a nucleotide sequence which has at least 95% identity, and optionally at least 98% identity, to SEQ ID NO: 3;
and/or
e. the core nucleotide sequence comprises or consists of a nucleotide sequence which has at least 95% identity, and optionally at least 98% identity, to SEQ ID NO: 3;
and/or
f. the core nucleotide sequence is identical to SEQ ID NO: 3.

9. The transcription regulatory element as claimed in claim 1 or in any one of claims 3 to 7, which comprises or consists of a nucleotide sequence which has a nucleotide sequence that has at least 90% identity, optionally at least 95% identity or optionally at least 98% identity to a nucleotide sequence selected from the group consisting of:
a. SEQ ID NO: 24;
b. SEQ ID NO: 25;
c. SEQ ID NO: 26;
d. SEQ ID NO: 27;
e. SEQ ID NO: 28; and
f. SEQ ID NO: 29.

10. The transcription regulatory element as claimed in any one of the preceding claims, wherein the transcription regulatory element comprises a promoter; optionally wherein the transcription regulatory element further comprises an enhancer; and optionally wherein the promoter is liver-specific.

11. A polynucleotide comprising a transcription regulatory element as claimed in any one of the preceding claims, wherein the transcription regulatory element is operably linked to a transgene, optionally wherein the transgene encodes a human protein.

12. The transcription regulatory element as claimed in any one of claims 1 to 10, wherein the transcription regulatory element is part of a vector comprising a transgene, optionally wherein the transgene encodes a human protein, and optionally wherein the vector is a viral particle such as an AAV vector.

13. The polynucleotide as claimed in claim 11 or the transcription regulatory element as claimed in claim 12, wherein:
a. the transcription regulatory element expresses the transgene at 50% or better compared to a transcription regulatory element defined by SEQ ID NO: 1 or SEQ ID NO: 33;
b. the transcription regulatory element expresses the transgene at 80% or better compared to a transcription regulatory element defined by SEQ ID NO: 1 or SEQ ID NO: 33; or
c. the transcription regulatory element expresses the transgene at 100% or better compared to a transcription regulatory element defined by SEQ ID NO: 1 or SEQ ID NO: 33; or
d. the transcription regulatory element expresses the transgene at 150% or better compared to a transcription regulatory element defined by SEQ ID NO: 1 or SEQ ID NO: 33;
and optionally wherein expression of the transgene is determined in vitro in Huh7 cells.

14. The polynucleotide or transcription regulatory element as claimed in any one of claims 11 to 13, wherein:
a. the transgene encodes a protein or a non-translated RNA which optionally is an siRNA, or an miRNA, or a snRNA, or an antisense RNA;
and/or
b. the transgene is longer than 4 k nucleotides; optionally wherein the transgene is longer than 4.2 nucleotides;
and/or
c. the transgene is shorter than 4.5 k nucleotides, optionally wherein the transgene is shorter than 4.4 k nucleotides;
and/or
d. the transgene encodes FVIII; optionally wherein the transgene encodes a truncated or modified FVIII; optionally wherein the transgene encodes a B-domain deleted FVIII.

15. A vector comprising a nucleotide sequence which comprises: (i) the transcription regulatory element as claimed in any one of claims 1 to 10; and (ii) a transgene; and optionally wherein:
a. the vector nucleotide sequence further comprises a nucleotide sequence encoding a signal peptide; optionally wherein the nucleotide sequence encoding the signal peptide is 50 to 100 nucleotides in length; optionally wherein the nucleotide sequence encoding the signal peptide is shorter than 80 nucleotides;
and/or
b. the vector is a viral particle such as an AAV vector;
and/or
c. the transcription regulatory element expresses the transgene at 50% or better compared to a transcription regulatory element defined by SEQ ID NO: 1 or SEQ ID NO: 33; optionally wherein expression of the transgene is determined in vitro in Huh7 cells;
and/or
d. the transcription regulatory element expresses the transgene at 80% or better compared to a transcription regulatory element defined by SEQ ID NO: 1 or SEQ ID NO: 33; optionally wherein expression of the transgene is determined in vitro in Huh7 cells;
and/or
e. the transcription regulatory element expresses the transgene at 100% or better compared to a transcription regulatory element defined by SEQ ID NO: 1 or SEQ ID NO: 33; optionally wherein expression of the transgene is determined in vitro in Huh7 cells;
and/or
f. the transcription regulatory element expresses the transgene at 150% or better compared to a transcription regulatory element defined by SEQ ID NO: 1 or SEQ ID NO: 33; optionally wherein expression of the transgene is determined in vitro in Huh7 cells;
and/or
g. the transgene encodes a protein or a non-translated RNA which optionally is an siRNA, or an miRNA, or a snRNA, or an antisense RNA;
and/or
h. the transgene is longer than 4 k nucleotides; optionally wherein the transgene is longer than 4.2 nucleotides;
and/or
i. the transgene is shorter than 4.5 k nucleotides, optionally wherein the transgene is shorter than 4.4 k nucleotides;
and/or
j. the transgene encodes FVIII; optionally wherein the transgene encodes a truncated or modified FVIII; optionally wherein the transgene encodes a B-domain deleted FVIII;
and/or
k. the vector genome is shorter than 4.9 k nucleotides, and optionally wherein the vector genome is no shorter than 4.5 k nucleotides;
and/or
l. the vector genome is around 4.7 k nucleotides in length.

16. The vector as claimed in claim 15 for use in a method of treatment, optionally wherein the method of treatment is a method of gene therapy and/or a method of treating Haemophilia A.
